(19) United States
(12) Patent Application Publication
(10) Pub. No.: US 2023/0167384 A1
(43) Pub. Date: Jun. 1, 2023

(54) CLEANING COMPOSITIONS COMPRISING POLYPEPTIDES HAVING FRUCTAN DEGRADING ACTIVITY

(71) Applicant: Novozymes A/S, Bagsvaerd (DK)

(72) Inventors: Marc Dominique Morant, Frederiksberg (DK); Lorena Gonzalez Palmen, Akarp (SE); Klaus Gori, Dyssegaard (DK); Jesper Salomon, Holte (DK)

(73) Assignee: Novozymes A/S, Bagsvaerd (DK)

(21) Appl. No.: 17/913,103

(22) PCT Filed: Apr. 20, 2021
(86) PCT No.: PCT/EP2021/060242

§ 371 Date: Sep. 20, 2022

(30) Foreign Application Priority Data
Apr. 21, 2020 (EP) 20170628.0
Aug. 24, 2020 (EP) 20192387.7
Oct. 30, 2020 (EP) 20204923.5

(51) Int. Cl. C11D 3/386; C11D 3/395; C11D 3/37

(52) U.S. Cl. CPC C11D 3/38636; C11D 3/3951; C11D 3/37

(57) ABSTRACT

The invention relates to cleaning compositions comprising a fructan degrading enzyme and at least one cleaning component, and use of such compositions for cleaning of an item such as a textile or a surface.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to compositions, in particular cleaning compositions, comprising polypeptides having fructan degrading activity. The invention further relates to novel fructan degrading polypeptides, polynucleotides encoding the polypeptides, use of polypeptides having fructan degrading activity in cleaning processes and/or for removal or reduction of bacterial-derived fructan, and methods for removal or reduction of fructan. The invention further relates to nucleic acid constructs, vectors, and host cells comprising polynucleotides encoding the polypeptides as well as methods of producing and using the polypeptides.

### Description of the Related Art

Enzymes have been used in detergents for decades. Often a cocktail of various enzymes is added to detergent compositions, the enzyme cocktail comprising two or more enzymes that each target a specific substrate, e.g. amylases are active towards starch stains, proteases on protein stains and so forth. Textiles and surfaces such as laundry and dishes become soiled with many different types of soiling. The soiling may be composed of proteins, grease, starch etc. Complex stains composed of different organic materials such as food stains, sebum, dead cell material, and extracellular polymeric matrix (EPS) from e.g. biofilm are difficult to remove completely with traditional automatic dishwashing (ADW) and laundry detergent compositions.

One component that may contribute to the organic matter is fructan originating from bacteria, which are present in high numbers in laundry items. When the bacteria lyse, one of the components left in the textiles or on hard surfaces such as the inner surfaces of a washing machine is fructan from the destroyed cells. This fructan substrate may be sticky or gluing, which when present on textile attracts soils and may cause redeposition or backstaining of soil, resulting in a greying of the textile. Also, malodors from e.g. sweat, cigarette smoke and pollution are particularly difficult to remove from e.g. textiles. Malodor is a growing problem, particularly in laundry, with the changed habits of lower temperature washing, front-loading wash machines that save water but leave behind residual water between loads, thus allowing bacterial biofilms to flourish, line drying clothes to save energy rather than appliance drying, and the increased popularity of synthetic fabrics, such as athletic wear, that may retain odors more than natural fabrics. In conventional detergent compositions such as laundry detergents the above problems are often solved by adding perfumes. This solution is not completely effective, however, as it is short term and furthermore only serves to mask malodor rather than dealing with the underlying cause of malodor. There is thus a need in the art for new solutions for overcoming the problems of malodor and redeposition.

### SUMMARY OF THE INVENTION

The invention relates to cleaning compositions comprising a fructan degrading enzyme and at least one cleaning component.

The invention further relates to the use of such compositions for cleaning of an item such as a textile or a surface. The invention further relates to a method of cleaning an item, comprising contacting the item with a solution comprising a fructan degrading enzyme of the invention and at least one cleaning component. The invention further relates to compositions comprising a fructan degrading enzyme and at least one nuclease polypeptide having DNase or RNase activity, and use thereof for cleaning.

### Overview of Sequences

SEQ ID NO: 1 DNA encoding full length polypeptide from Paenibacillus amylolyticus
SEQ ID NO: 2 polypeptide derived from SEQ ID NO: 1
SEQ ID NO: 3 mature polypeptide obtained from Paenibacillus amylolyticus
SEQ ID NO: 4 DNA encoding full length polypeptide from Aspergillus deflectus
SEQ ID NO: 5 polypeptide derived from SEQ ID NO: 4
SEQ ID NO: 6 mature polypeptide obtained from Aspergillus deflectus
SEQ ID NO: 7 DNA encoding full length polypeptide from Aspergillus niger
SEQ ID NO: 8 polypeptide derived from SEQ ID NO: 7
SEQ ID NO: 9 mature polypeptide obtained from Aspergillus niger
SEQ ID NO: 10 DNA encoding full length polypeptide from Penicillium ochrochloron
SEQ ID NO: 11 polypeptide derived from SEQ ID NO: 10
SEQ ID NO: 12 mature polypeptide obtained from Penicillium ochrochloron
SEQ ID NO: 13 DNA encoding full length polypeptide from Penicillium murcianum
SEQ ID NO: 14 polypeptide derived from SEQ ID NO: 13
SEQ ID NO: 15 mature polypeptide obtained from Penicillium murcianum
SEQ ID NO: 16 DNA encoding full length polypeptide from Bacillus licheniformis
SEQ ID NO: 17 polypeptide derived from SEQ ID NO: 16
SEQ ID NO: 18 mature polypeptide obtained from Bacillus licheniformis
SEQ ID NO: 19 mature DNase polypeptide obtained from Bacillus cibi
SEQ ID NO: 20 mature DNase polypeptide obtained from Bacillus licheniformis
SEQ ID NO: 21 mature DNase polypeptide obtained from Bacillus subtilis
SEQ ID NO: 22 mature DNase polypeptide obtained from Aspergillus oryzae
SEQ ID NO: 23 mature DNase polypeptide obtained from Trichoderma harzianum
SEQ ID NO: 24 motif [WPG][GMTH][NH][AEILDNMV]
SEQ ID NO: 25 motif A[YF]S[LN]D[QK]
SEQ ID NO: 26 motif AYSNDK
SEQ ID NO: 27 motif [HS]WGH
SEQ ID NO: 28 motif [QLKEYMRVTANI][NSYHATRD][WF][MEVKTL][NGA][EVDLIH][PAEHDS][NATSKGVERQCD]
SEQ ID NO: 29 motif KNWMNEPN
SEQ ID NO: 30 motif WMN[DE]
SEQ ID NO: 31 motif WMN[DE]PNG
SEQ ID NO: 32 motif W[GMT]N[NDEI]

### Definitions

Fructan degrading enzymes: The term “fructan degrading enzyme” means an enzyme having activity towards fructan. The enzyme may also be referred to herein as a “fructanase” or a polypeptide or enzyme having fructan degrading activity or fructanase activity.

Fructan is a polymer of fructose molecules that is found in certain classes of Gram-positive and Gram-negative bacteria, for example in Bacillus, Streptococcus, Pseudomonas, Erwinia and Actinomyces, as well as in some fungi, for example Aspergillus and Penicillium. Fructan molecules produced by bacteria consist mainly of β(2,6)-linked fructosyl residues and some β(2,1)-linked branches. Such fructans are called levans and can reach a degree of polymerization (DP) of more than 100,000 fructosyl units. (Vijn and Smeekens, Plant Physiology 1999, Vol. 120: 351-359; Van den Ende, J Exp Bot. 2018, Vol. 69 (18): 4227-4231). Another major type of fructan, inulin, comprises primarily β-(2,1) linked fructosyl residues and some β-(2,6) linked branches.

The glycosyl hydrolase 32 (GH32) family contains enzymes that hydrolyze fructose-containing polysaccharides. The GH32 family includes inulinases (EC 3.2.1.7) and exo-inulinases (fructan β-fructosidases) (EC 3.2.1.80), levanases (EC 3.2.1.65), 2,6-β-fructan 6-levanbiohydrolases (EC 3.2.1.64), fructan β-(2,1)-fructosidases (1-fructan exohydrolases) (EC 3.2.1.153) and fructan β-(2,6)-fructosidases (EC 3.2.1.154). Other GH32 family enzymes include those displaying transglycosylating activities such as sucrose:sucrose 1-fructosyltransferases (EC 2.4.1.99), fructan:fructan 1-fructosyltransferases (2,1-fructan:2,1-fructan 1-fructosyltransferases) (EC 2.4.1.100), sucrose:fructan 6-fructosyltransferases (EC 2.4.1.10), fructan:fructan 6G-fructosyltransferases (EC 2.4.1.243) and levan fructosyltransferases (EC 2.4.1.-). (Mirjam Czjzek and Wim Van den Ende, “Glycoside Hydrolase Family 32” in CAZypedia, available at http://www.cazypedia.org/, accessed 14 Feb. 2020).

The enzymes used in the compositions of the invention are thus from the GH32 family and may be referred to as GH32 fructanases or GH32 fructan degrading enzymes or polypeptides. The enzymes may have endo or exo activity.

In preferred embodiments, the GH32 fructanases comprise one or more of the fructanase activities mentioned above, for example one or more of fructan β(2,1)-fructosidase activity (EC 3.2.1.153), fructan β(2,6)-fructosidase activity (EC 3.2.1.154), fructan β-fructosidase activity (EC 3.2.1.80), levanase activity (EC 3.2.1.65), 2,6-β-fructan 6-levanbiohydrolase activity (EC 3.2.1.64) and inulinase activity (EC 3.2.1.7).

For purposes of the present invention, fructan degrading activity may be determined according to the method described below in Example 2 below.

A biofilm is organic matter produced by any group of microorganisms in which cells stick to each other or stick to a surface, such as a textile, dishware or hard surface or another kind of surface. These adherent cells are frequently embedded within a self-produced matrix of extracellular polymeric substance (EPS). Biofilm EPS is a polymeric conglomeration generally composed of extracellular DNA, proteins, and polysaccharides. Biofilms may form on living or non-living surfaces. The microbial cells growing in a biofilm are physiologically distinct from planktonic cells of the same organism, which, by contrast, are single cells that may float or swim in a liquid medium. Bacteria living in a biofilm usually have significantly different properties from planktonic bacteria of the same species, as the dense and protected environment of the film allows them to cooperate and interact in various ways. One benefit of this environment for the microorganisms is increased resistance to detergents and antibiotics, as the dense extracellular matrix and the outer layer of cells protect the interior of the community. The biofilm living bacteria do not lose their ability to live as planktonic cells if the biofilm matrix is compromised. On laundry, biofilm- or EPS-producing bacteria can be found among the following species: Acinetobacter sp., Aeromicrobium sp., Brevundimonas sp., Microbacterium sp., Micrococcus luteus, Pseudomonas sp., Staphylococcus epidermidis, and Stenotrophomonas sp.

The term “catalytic domain” means the region of an enzyme containing the catalytic machinery of the enzyme.

The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

The term “clade” means a group of polypeptides clustered together on the basis of homologous features traced to a common ancestor. Polypeptide clades can be visualized as phylogenetic trees and a clade is a group of polypeptides that consists of a common ancestor and all its lineal descendants.

The term “coding sequence” means a polynucleotide which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

The term “cleaning component” (or “detergent component”) means a detergent adjunct ingredient that is different from the polypeptides of this invention. The precise nature of these additional cleaning or adjunct components, and levels of incorporation thereof, will depend on the physical form of the composition and the nature of the operation for which it is to be used. Suitable cleaning components include, but are not limited to the components described below, such as surfactants, builders and co-builders, flocculating aid, chelating agents, dye transfer inhibitors, enzymes (other than the enzymes of the invention), enzyme stabilizers, enzyme inhibitors, catalytic materials, bleach activators, hydrogen peroxide, sources of hydrogen peroxide, preformed peracids, polymeric agents, clay soil removal/anti-redeposition agents, brighteners, suds suppressors, dyes, perfumes, structure elasticizing agents, fabric softeners, carriers, hydrotropes, fabric hueing agents, anti-foaming agents, dispersants, processing aids, and/or pigments. Cleaning compositions will typically contain at least one surfactant along with additional components such as at least one builder and/or at least one bleach component

The term “cleaning composition” includes “detergent composition” and refers to compositions that find use in the removal of undesired compounds from items to be cleaned, such as textiles. The detergent composition may be used to e.g. clean textiles for both household cleaning and industrial cleaning. The term encompasses any materials/compounds selected for the particular type of cleaning composition desired and the form of the product (e.g., liquid, gel, powder, granulate, paste, or spray compositions) and includes, but is not limited to, detergent compositions such as liquid and/or solid laundry detergents and fine fabric detergents; fabric fresheners; fabric softeners; and textile and laundry pre-spotters/pretreatment. In addition to containing the enzyme of the invention, the detergent formulation may contain one or more additional enzymes (such as proteases, amylases, lipases, cutinases, cellulases, endoglucanases, xyloglucanases, pectinases, pectin lyases, xanthanases, peroxidases, haloperoxygenases, catalases, mannanases, nucleases or any mixture thereof), and/or detergent adjunct ingredients such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferases, hydrolytic enzymes, oxido reductases, bluing agents and fluorescent dyes, antioxidants, and solubilizers.

The term “expression” includes any step involved in the production of a polypeptide including, but not limited to, transcription, post-transcriptional modification, translation, post-translational modification, and secretion.

The term “expression vector” means a linear or circular DNA molecule that comprises a polynucleotide encoding a polypeptide and is operably linked to control sequences that provide for its expression.

The term “fragment” means a polypeptide having one or more amino acids absent from the amino and/or carboxyl terminus of a mature polypeptide or domain, where the fragment has fructan degrading activity.

The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance). An isolated substance may be present in a fermentation broth sample; e.g. a host cell may be genetically modified to express the polypeptide of the invention. The fermentation broth from that host cell will comprise the isolated polypeptide. It will be apparent to persons skilled in the art that the polypeptides disclosed herein are preferably in isolated form.

The term “laundering” relates to both household laundering and industrial laundering and means the process of treating textiles with a solution containing a cleaning or detergent composition of the present invention. The laundering process can for example be carried out using e.g. a household or an industrial washing machine or can be carried out by hand.

The term “malodor” means an odor which is not desired on clean items. The cleaned item should smell fresh and clean without malodors adhered to the item. One example of malodor is compounds with an unpleasant smell, which may be produced by microorganisms. Another example is unpleasant smells which can be sweat or body odor adhered to an item which has been in contact with human or animal. Another example of malodor can be the odor from spices, which sticks to items for example curry or other spices which smell strongly.

The term “mature polypeptide” means a polypeptide in its mature form following N terminal processing (e.g., removal of signal peptide).

In one aspect, the mature polypeptide is amino acids 1 to 892 of SEQ ID NO: 2. Amino acids −31 to −1 of SEQ ID NO: 2 are a signal peptide.

In one aspect, the mature polypeptide is amino acids 1 to 496 of SEQ ID NO: 5. Amino acids −16 to −1 of SEQ ID NO: 5 are a signal peptide.

In one aspect, the mature polypeptide is amino acids 1 to 491 of SEQ ID NO: 8. Amino acids −25 to −1 of SEQ ID NO: 8 are a signal peptide.

In one aspect, the mature polypeptide is amino acids 1 to 1411 of SEQ ID NO: 11. Amino acids −21 to −1 of SEQ ID NO: 11 are a signal peptide.

In one aspect, the mature polypeptide is amino acids 1 to 492 of SEQ ID NO: 14. Amino acids −15 to −1 of SEQ ID NO: 14 are a signal peptide.

In one aspect, the mature polypeptide is amino acids 1 to 653 of SEQ ID NO: 17. Amino acids −24 to −1 of SEQ ID NO: 17 are a signal peptide.

It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

The term “mature polypeptide coding sequence” means a polynucleotide that encodes a mature polypeptide having fructan degrading activity.

In one aspect, the mature polypeptide coding sequence is nucleotides 94 to 2769 of SEQ ID NO: 1 and nucleotides 1 to 93 of SEQ ID NO: 1 encode a signal peptide.

In one aspect, the mature polypeptide coding sequence is nucleotides 49 to 1536 of SEQ ID NO: 4 and nucleotides 1 to 48 of SEQ ID NO: 4 encode a signal peptide.

In one aspect, the mature polypeptide coding sequence is nucleotides 76 to 1548 of SEQ ID NO: 7 and nucleotides 1 to 75 of SEQ ID NO: 7 encode a signal peptide.

In one aspect, the mature polypeptide coding sequence is nucleotides 64 to 399 and 453 to 4349 of SEQ ID NO: 10, and nucleotides 1 to 63 of SEQ ID NO: 10 encode a signal peptide.

In one aspect, the mature polypeptide coding sequence is nucleotides 46 to 1521 of SEQ ID NO: 13 and nucleotides 1 to 45 of SEQ ID NO: 13 encode a signal peptide.

In one aspect, the mature polypeptide coding sequence is nucleotides 73 to 2031 of SEQ ID NO: 16 and nucleotides 1 to 72 of SEQ ID NO: 16 encode a signal peptide.

The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

The term “operably linked” means a configuration in which a control sequence is placed at an appropriate position relative to the coding sequence of a polynucleotide such that the control sequence directs expression of the coding sequence.

The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Alignment−Total Number of Gaps in Alignment)

The term “variant” means a polypeptide having fructan degrading activity comprising an alteration, i.e., a substitution, insertion, and/or deletion, at one or more positions. A substitution means replacement of the amino acid occupying a position with a different amino acid; a deletion means removal of the amino acid occupying a position; and an insertion means adding an amino acid adjacent to and immediately following the amino acid occupying a position.

Nomenclature: For purposes of the present invention, the nomenclature [E/Q] or [EQ] means that the amino acid at this position may be a glutamic acid (Glu, E) or a glutamine (Gln, Q). Likewise, the nomenclature [V/G/A/l] or [VGAI] means that the amino acid at this position may be a valine (Val, V), glycine (Gly, G), alanine (Ala, A) or isoleucine (Ile, I), and so forth for other combinations as described herein. Unless otherwise limited further, the amino acid X is defined such that it may be any of the 20 natural amino acids.

### DETAILED DESCRIPTION OF THE INVENTION

As mentioned in the background section above, textiles and surfaces such as laundry and dishes may become soiled with many different types of soiling. A single complex stain such as a food stain, sebum, dead cells debris, EPS or biofilm related stains is often composed of different organic material such as proteins, polysaccharides, grease etc., which are often difficult to remove completely with traditional detergent compositions. Further, such stains may give rise to disadvantages such as redeposition or malodor. The polypeptides of the invention address this problem, providing good cleaning effects on complex stains such as biofilm and EPS as well as reduced redeposition and malodor from e.g. textiles and tableware.

The polypeptides of the invention are fructan degrading enzymes having one or more of the enzyme activities listed above, e.g. one or more of fructan β(2,1)-fructosidase activity (EC 3.2.1.153), fructan β(2,6)-fructosidase activity (EC 3.2.1.154), fructan R-fructosidase activity (EC 3.2.1.80), levanase activity (EC 3.2.1.65), 2,6-β-fructan 6-levanbiohydrolase activity (EC 3.2.1.64) and inulinase activity (EC 3.2.1.7). Preferred activities include β(2,6)-fructosidase activity (EC 3.2.1.154) and/or fructan β(2,1)-fructosidase activity (EC 3.2.1.153), and preferably at least β(2,6)-fructosidase activity.

The polypeptides of the invention comprise a GH32 domain, as defined in CAZY (Lombard, Henrissat et al, 2014. The carbohydrate-active enzymes database (CAZy), in 2013, Nucleic Acids Res. 42, http://www.cazy.org/). Preferably, the polypeptides also contain a glycosyl hydrolase family 32 C terminal domain (“GH32C domain”), as defined by Pfam domain ID PF08244 (The Pfam protein families database: towards a more sustainable future, Finn et al., Nucleic Acids Research (2016) Database Issue 44:D279-D285).

Also, clusters or clades are described herein, defined by specific motifs shared by the polypeptides of the specific clades. Construction of a phylogenetic tree and identification of clades and clusters are described in Example 3.

### Polypeptides Having Fructan Degrading Activity

One embodiment of the invention relates to a fructan degrading polypeptide, i.e. a GH32 fructanase having one or more of the enzyme activities described above, as well as cleaning compositions comprising the fructan degrading enzyme and use thereof for cleaning. In one embodiment, the fructan degrading polypeptide is a polypeptide that belongs to the WMNE clade and comprises the motif [WPG][GMTH][NH][AEILDNMV] (SEQ ID NO: 24), preferably the motif W[GMT]N[NDEI] (SEQ ID NO: 32).

In another embodiment, the fructan degrading enzyme belongs to the AYSN clade and comprises the motif A[YF]S[LN]D[QK] (SEQ ID NO: 25) and/or the motif [HS]WGH (SEQ ID NO: 27).

In a further embodiment, the fructan degrading enzyme belongs to the WMNEP clade and comprises the motif [QLKEYMRVTANI][NSYHATRD] [WF][MEVKTL] [NGA] [EVDLIH] [PAEHDS][NATSKGVERQCD] (SEQ ID NO: 28).

The motif of SEQ ID NO: 24 preferably has the sequence W[GMT]N[NDEI] (SEQ ID NO: 32), more particularly be WMN[DE] (SEQ ID NO: 30), for example WMN[DE]PNG (SEQ ID NO: 31).

In an embodiment, the present invention relates to polypeptides having a sequence identity to the mature polypeptide of SEQ ID NO: 5 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have fructan degrading activity, as well as use thereof in compositions and cleaning methods of the invention. In one aspect, the polypeptides differ by up to 10 amino acids, e.g., 1, 2, 3, 4, 5, 6, 7, 8, 9, or 10, from the mature polypeptide of SEQ ID NO: 5. The polypeptide may also be a fragment of the mature polypeptide of SEQ ID NO: 5 which has fructan degrading activity. The polypeptide preferably belongs to the WMNE clade and comprises the motif [WPG][GMTH][NH][AEILDNMV] (SEQ ID NO: 24), preferably the motif W[GMT]N[NDEI] (SEQ ID NO: 32), and optionally also belongs to the AYSN clade and comprises the motif A[YF]S[LN]D[QK](SEQ ID NO: 25) and/or the motif [HS]WGH (SEQ ID NO: 27) and/or belongs to the WMNEP clade and comprises the motif [QLKEYMRVTANI] [NSYHATRD][WF][MEVKTL] [NGA] [EVDLIH][PAEHDS] [NATSKGVERQCD] (SEQ ID NO: 28).

In an embodiment, the present invention relates to polypeptides having a sequence identity to the mature polypeptide of SEQ ID NO: 8 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have fructan degrading activity, as well as use thereof in compositions and cleaning methods of the invention. In one aspect, the polypeptides differ by up to 10 amino acids, e.g., 1, 2, 3, 4, 5, 6, 7, 8, 9, or 10, from the mature polypeptide of SEQ ID NO: 8. The polypeptide may also be a fragment of the mature polypeptide of SEQ ID NO: 8 which has fructan degrading activity. The polypeptide preferably belongs to the WMNE clade and comprises the motif [WPG][GMTH][NH][AEILDNMV] (SEQ ID NO: 24), preferably the motif W[GMT]N[NDEI] (SEQ ID NO: 32), and optionally also belongs to the AYSN clade and comprises the motif A[YF]S[LN]D[QK](SEQ ID NO: 25) and/or the motif [HS]WGH (SEQ ID NO: 27) and/or belongs to the WMNEP clade and comprises the motif [QLKEYMRVTANI] [NSYHATRD][WF][MEVKTL] [NGA] [EVDLIH][PAEHDS] [NATSKGVERQCD] (SEQ ID NO: 28).

In an embodiment, the present invention relates to polypeptides having a sequence identity to the mature polypeptide of SEQ ID NO: 11 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have fructan degrading activity, as well as use thereof in compositions and cleaning methods of the invention. In one aspect, the polypeptides differ by up to 10 amino acids, e.g., 1, 2, 3, 4, 5, 6, 7, 8, 9, or 10, from the mature polypeptide of SEQ ID NO: 11. The polypeptide may also be a fragment of the mature polypeptide of SEQ ID NO: 11 which has fructan degrading activity. The polypeptide preferably belongs to the WMNE clade and comprises the motif [WPG][GMTH][NH][AEILDNMV] (SEQ ID NO: 24), preferably the motif W[GMT]N[NDEI] (SEQ ID NO: 32), and optionally also belongs to the AYSN clade and comprises the motif A[YF]S[LN]D[QK](SEQ ID NO: 25) and/or the motif [HS]WGH (SEQ ID NO: 27) and/or belongs to the WMNEP clade and comprises the motif [QLKEYMRVTANI] [NSYHATRD][WF][MEVKTL] [NGA] [EVDLIH][PAEHDS] [NATSKGVERQCD] (SEQ ID NO: 28).

In an embodiment, the present invention relates to polypeptides having a sequence identity to the mature polypeptide of SEQ ID NO: 14 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have fructan degrading activity, as well as use thereof in compositions and cleaning methods of the invention. In one aspect, the polypeptides differ by up to 10 amino acids, e.g., 1, 2, 3, 4, 5, 6, 7, 8, 9, or 10, from the mature polypeptide of SEQ ID NO: 14. The polypeptide may also be a fragment of the mature polypeptide of SEQ ID NO: 14 which has fructan degrading activity. The polypeptide preferably belongs to the WMNE clade and comprises the motif [WPG][GMTH][NH][AEILDNMV] (SEQ ID NO: 24), preferably the motif W[GMT]N[NDEI] (SEQ ID NO: 32), and optionally also belongs to the AYSN clade and comprises the motif A[YF]S[LN]D[QK](SEQ ID NO: 25) and/or the motif [HS]WGH (SEQ ID NO: 27) and/or belongs to the WMNEP clade and comprises the motif [QLKEYMRVTANI] [NSYHATRD][WF][MEVKTL] [NGA] [EVDLIH][PAEHDS] [NATSKGVERQCD] (SEQ ID NO: 28).

In an embodiment, the present invention relates to polypeptides having a sequence identity to the mature polypeptide of SEQ ID NO: 17 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have fructan degrading activity, as well as use thereof in compositions and cleaning methods of the invention. In one aspect, the polypeptides differ by up to 10 amino acids, e.g., 1, 2, 3, 4, 5, 6, 7, 8, 9, or 10, from the mature polypeptide of SEQ ID NO: 17. The polypeptide may also be a fragment of the mature polypeptide of SEQ ID NO: 17 which has fructan degrading activity. The polypeptide preferably belongs to the WMNE clade and comprises the motif [WPG][GMTH][NH][AEILDNMV] (SEQ ID NO: 24), preferably the motif W[GMT]N[NDEI] (SEQ ID NO: 32), and optionally also belongs to the AYSN clade and comprises the motif A[YF]S[LN]D[QK](SEQ ID NO: 25) and/or the motif [HS]WGH (SEQ ID NO: 27) and/or belongs to the WMNEP clade and comprises the motif [QLKEYMRVTANI] [NSYHATRD][WF][MEVKTL] [NGA] [EVDLIH][PAEHDS] [NATSKGVERQCD] (SEQ ID NO: 28).

In a particular embodiment the invention relates to polypeptides having a sequence identity to the polypeptide of SEQ ID NO: 6 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has fructan degrading activity, as well as use thereof in compositions and cleaning methods of the invention. The polypeptide may for example comprise or consist of the amino acid sequence of SEQ ID NO: 6. The polypeptide may also be a fragment of SEQ ID NO: 6 which has fructan degrading activity. The polypeptide preferably belongs to the WMNE clade and comprises the motif [WPG][GMTH][NH][AEILDNMV] (SEQ ID NO: 24), preferably the motif W[GMT]N[NDEI] (SEQ ID NO: 32), and optionally also belongs to the AYSN clade and comprises the motif A[YF]S[LN]D[QK] (SEQ ID NO: 25) and/or the motif [HS]WGH (SEQ ID NO: 27) and/or belongs to the WMNEP clade and comprises the motif [QLKEYMRVTANI][NSYHATRD][WF][MEVKTL] [NGA] [EVDLIH] [PAEHDS] [NATSKGVERQCD] (SEQ ID NO: 28).

In a particular embodiment the invention relates to polypeptides having a sequence identity to the polypeptide of SEQ ID NO: 9 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has fructan degrading activity, as well as use thereof in compositions and cleaning methods of the invention. The polypeptide may for example comprise or consist of the amino acid sequence of SEQ ID NO: 9. The polypeptide may also be a fragment of SEQ ID NO: 9 which has fructan degrading activity. The polypeptide preferably belongs to the WMNE clade and comprises the motif [WPG][GMTH][NH][AEILDNMV] (SEQ ID NO: 24), preferably the motif W[GMT]N[NDEI] (SEQ ID NO: 32), and optionally also belongs to the AYSN clade and comprises the motif A[YF]S[LN]D[QK] (SEQ ID NO: 25) and/or the motif [HS]WGH (SEQ ID NO: 27) and/or belongs to the WMNEP clade and comprises the motif [QLKEYMRVTANI][NSYHATRD][WF][MEVKTL] [NGA] [EVDLIH] [PAEHDS] [NATSKGVERQCD] (SEQ ID NO: 28).

In a particular embodiment the invention relates to polypeptides having a sequence identity to the polypeptide of SEQ ID NO: 12 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has fructan degrading activity, as well as use thereof in compositions and cleaning methods of the invention. The polypeptide may for example comprise or consist of the amino acid sequence of SEQ ID NO: 12. The polypeptide may also be a fragment of SEQ ID NO: 12 which has fructan degrading activity. The polypeptide preferably belongs to the WMNE clade and comprises the motif [WPG][GMTH][NH][AEILDNMV] (SEQ ID NO: 24), preferably the motif W[GMT]N[NDEI] (SEQ ID NO: 32), and optionally also belongs to the AYSN clade and comprises the motif A[YF]S[LN]D[QK] (SEQ ID NO: 25) and/or the motif [HS]WGH (SEQ ID NO: 27) and/or belongs to the WMNEP clade and comprises the motif [QLKEYMRVTANI][NSYHATRD][WF][MEVKTL] [NGA] [EVDLIH] [PAEHDS] [NATSKGVERQCD] (SEQ ID NO: 28).

In a particular embodiment the invention relates to polypeptides having a sequence identity to the polypeptide of SEQ ID NO: 15 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has fructan degrading activity, as well as use thereof in compositions and cleaning methods of the invention. The polypeptide may for example comprise or consist of the amino acid sequence of SEQ ID NO: 15. The polypeptide may also be a fragment of SEQ ID NO: 15 which has fructan degrading activity. The polypeptide preferably belongs to the WMNE clade and comprises the motif [WPG][GMTH][NH][AEILDNMV] (SEQ ID NO: 24), preferably the motif W[GMT]N[NDEI] (SEQ ID NO: 32), and optionally also belongs to the AYSN clade and comprises the motif A[YF]S[LN]D[QK] (SEQ ID NO: 25) and/or the motif [HS]WGH (SEQ ID NO: 27) and/or belongs to the WMNEP clade and comprises the motif [QLKEYMRVTANI][NSYHATRD][WF][MEVKTL] [NGA] [EVDLIH] [PAEHDS] [NATSKGVERQCD] (SEQ ID NO: 28).

In a particular embodiment the invention relates to polypeptides having a sequence identity to the polypeptide of SEQ ID NO: 18 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has fructan degrading activity, as well as use thereof in compositions and cleaning methods of the invention. The polypeptide may for example comprise or consist of the amino acid sequence of SEQ ID NO: 18. The polypeptide may also be a fragment of SEQ ID NO: 18 which has fructan degrading activity. The polypeptide preferably belongs to the WMNE clade and comprises the motif [WPG][GMTH][NH][AEILDNMV] (SEQ ID NO: 24), preferably the motif W[GMT]N[NDEI] (SEQ ID NO: 32), and optionally also belongs to the AYSN clade and comprises the motif A[YF]S[LN]D[QK] (SEQ ID NO: 25) and/or the motif [HS]WGH (SEQ ID NO: 27) and/or belongs to the WMNEP clade and comprises the motif [QLKEYMRVTANI][NSYHATRD][WF][MEVKTL] [NGA] [EVDLIH] [PAEHDS] [NATSKGVERQCD] (SEQ ID NO: 28).

In any of the embodiments disclosed herein, the polypeptide has preferably been isolated, i.e. the polypeptide is in an “isolated” form or environment as defined above.

The polynucleotide of SEQ ID NO: 4, SEQ ID NO: 7, SEQ ID NO: 10, SEQ ID NO: 13 or SEQ ID NO: 16 or a subsequence thereof, as well as a fragment thereof may be used to design nucleic acid probes to identify and clone DNA encoding polypeptides having fructan degrading activity from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having fructan degrading activity. Genomic or other DNA from such other strains may be separated by agarose or polyacrylamide gel electrophoresis, or other separation techniques. DNA from the libraries or the separated DNA may be transferred to and immobilized on nitrocellulose or another suitable carrier material. In order to identify a clone or DNA that hybridizes with SEQ ID NO: 4, SEQ ID NO: 7, SEQ ID NO: 10, SEQ ID NO: 13 or SEQ ID NO: 16, the carrier material is used in a Southern blot.

For purposes of the present invention, hybridization indicates that the polynucleotide hybridizes to a labeled nucleic acid probe corresponding to (i) SEQ ID NO: 4, SEQ ID NO: 7, SEQ ID NO: 10, SEQ ID NO: 13 or SEQ ID NO: 16; (ii) the mature polypeptide coding sequence of SEQ ID NO: 4, SEQ ID NO: 7, SEQ ID NO: 10, SEQ ID NO: 13 or SEQ ID NO: 16; (iii) the full-length complement thereof; or (iv) a subsequence thereof, under standard low to high stringency conditions. Molecules to which the nucleic acid probe hybridizes under these conditions can be detected using, for example, X-ray film or any other detection means known in the art.

In another embodiment, the present invention relates to a polypeptide having fructan degrading activity encoded by a polynucleotide having a sequence identity to the mature polypeptide coding sequence of SEQ ID NO: 4 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having fructan degrading activity encoded by a polynucleotide having a sequence identity to the mature polypeptide coding sequence of SEQ ID NO: 7 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having fructan degrading activity encoded by a polynucleotide having a sequence identity to the mature polypeptide coding sequence of SEQ ID NO: 10 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having fructan degrading activity encoded by a polynucleotide having a sequence identity to the mature polypeptide coding sequence of SEQ ID NO: 13 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having fructan degrading activity encoded by a polynucleotide having a sequence identity to the mature polypeptide coding sequence of SEQ ID NO: 16 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to variants of the polypeptide shown in SEQ ID NO: 3 comprising a substitution, deletion, and/or insertion at one or more positions. In an embodiment, the number of amino acid substitutions, deletions and/or insertions introduced into the polypeptide shown SEQ ID NO: 3 is up to 10, e.g., 1, 2, 3, 4, 5, 6, 7, 8, 9, or 10.

In another embodiment, the present invention relates to variants of the polypeptide shown in SEQ ID NO: 6 comprising a substitution, deletion, and/or insertion at one or more positions. In an embodiment, the number of amino acid substitutions, deletions and/or insertions introduced into the polypeptide shown SEQ ID NO: 6 is up to 10, e.g., 1, 2, 3, 4, 5, 6, 7, 8, 9, or 10.

In another embodiment, the present invention relates to variants of the polypeptide shown in SEQ ID NO: 9 comprising a substitution, deletion, and/or insertion at one or more positions. In an embodiment, the number of amino acid substitutions, deletions and/or insertions introduced into the polypeptide shown SEQ ID NO: 9 is up to 10, e.g., 1, 2, 3, 4, 5, 6, 7, 8, 9, or 10.

In another embodiment, the present invention relates to variants of the polypeptide shown in SEQ ID NO: 12 comprising a substitution, deletion, and/or insertion at one or more positions. In an embodiment, the number of amino acid substitutions, deletions and/or insertions introduced into the polypeptide shown SEQ ID NO: 12 is up to 10, e.g., 1, 2, 3, 4, 5, 6, 7, 8, 9, or 10.

In another embodiment, the present invention relates to variants of the polypeptide shown in SEQ ID NO: 15 comprising a substitution, deletion, and/or insertion at one or more positions. In an embodiment, the number of amino acid substitutions, deletions and/or insertions introduced into the polypeptide shown SEQ ID NO: 15 is up to 10, e.g., 1, 2, 3, 4, 5, 6, 7, 8, 9, or 10.

In another embodiment, the present invention relates to variants of the polypeptide shown in SEQ ID NO: 18 comprising a substitution, deletion, and/or insertion at one or more positions. In an embodiment, the number of amino acid substitutions, deletions and/or insertions introduced into the polypeptide shown SEQ ID NO: 18 is up to 10, e.g., 1, 2, 3, 4, 5, 6, 7, 8, 9, or 10.

The amino acid changes in any of the embodiments above or elsewhere herein may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tag, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for fructan degradation activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7:127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The polypeptide may be a hybrid polypeptide in which a region of one polypeptide is fused at the N-terminus or the C-terminus of a region of another polypeptide.

The polypeptide may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

### Sources of Polypeptides Having Fructan Degrading Activity

A polypeptide having fructan degrading activity of the present invention may be obtained from microorganisms of any genus, in particular a bacterial or fungal genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly, e.g. from a bacterium.

The fructan degrading polypeptide is preferably of microbial origin, for example of bacterial or fungal origin. The term “origin” in this context should be understood to mean not only polypeptides that are obtained from the microbial, e.g. bacterial or fungal, source as such, but also chemically modified mutants or protein engineered variants. Those skilled in the art will be aware that protein engineering is commonly used to modify one or more amino acid residues in parent enzymes, e.g. wildtype enzymes, to improve one or more desired characteristics, for example enzyme activity, stability, expression level, etc.

In some aspects, the polypeptide may be obtained from a bacterial source. In one aspect, the polypeptide is an Alicyclobacillus polypeptide. In one aspect, the polypeptide is a Tumebacillus polypeptide. In one aspect, the polypeptide is a Halomonas polypeptide. In one aspect, the polypeptide is a Kribbella polypeptide, e.g., a polypeptide obtained from Kribbella aluminosa. In one aspect, the polypeptide is a Streptomyces polypeptide, e.g., a polypeptide obtained from Streptomyces griseus. In one aspect, the polypeptide is a Nonomuraea polypeptide, e.g., a polypeptide obtained from Nonomuraea coxensis, Nonomuraea dietziae or Nonomuraea guangzhouensis. In one aspect, the polypeptide is a Micromonospora polypeptide, e.g., a polypeptide obtained from Micromonospora peucetia, Micromonospora fulvopurpurea or Micromonospora maritima. In one aspect, the polypeptide is a Laceyella polypeptide, e.g., a polypeptide obtained from Laceyella sacchari. In one aspect, the polypeptide is a Bacillus polypeptide, e.g., a polypeptide obtained from Bacillus sporothermodurans or Bacillus cohnii. In one aspect, the polypeptide is a Lysobacter polypeptide, e.g., a polypeptide obtained from Lysobacter antibioticus or Lysobacter capsica. In one aspect, the polypeptide is a Hamadaea polypeptide, e.g., a polypeptide obtained from Hamadaea tsunoensis. In one aspect, the polypeptide is a Paenibacillus polypeptide, e.g., a polypeptide obtained from Paenibacillus pini. In one aspect, the polypeptide is a Thermostaphylospora polypeptide, e.g., a polypeptide obtained from Thermostaphylospora chromogena. In one aspect, the polypeptide is a Pseudomonas polypeptide, e.g., a polypeptide obtained from Pseudomonas peli or Pseudomonas pseudoalcaligenes.

In another aspect, the polypeptide may be obtained from a fungal source, for example from a fungus of the Aspergillaceae family, e.g. of the genus Aspergillus or Penicillium.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including variant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase I, Trichoderma reesei endoglucanase V, Trichoderma reesei xylanase I, Trichoderma reesei xylanase II, Trichoderma reesei xylanase Ill, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and variant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase Ill, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus nigerglucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosylaminoimidazole synthase), amdS (acetamidase), argB (ornithine carbamoyltransferase), bar (phosphinothricin acetyltransferase), hph (hygromycin phosphotransferase), niaD (nitrate reductase), niaA (nitrite reductase), pyrG (orotidine-5′phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location in a chromosome. To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMß1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a polypeptide of the present invention, comprising (a) cultivating a cell which in its wild-type form produces the polypeptide, under conditions conducive for production of the polypeptide; and optionally, (b) recovering the polypeptide.

The present invention also relates to recombinant methods of producing a polypeptide of the present invention, comprising (a) cultivating a recombinant host cell of the present invention capable of expressing the polypeptide under conditions conducive for production of the polypeptide; and optionally, (b) recovering the polypeptide.

One embodiment of the invention relates to a method of producing a polypeptide, wherein the polypeptide is selected from the group consisting of: SEQ ID NO: 3, SEQ ID NO: 6, SEQ ID NO: 9, SEQ ID NO: 12, SEQ ID NO: 15 and SEQ ID NO: 18, and polypeptides having at least 60% sequence identity, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity hereto, comprising (a) cultivating a recombinant host cell of the present invention capable of expressing one of the polypeptides under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid-state fermentations) in laboratory or industrial fermenters in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide. Another option is to use a supernatant in which the polypeptide has been expressed as a source of the polypeptide.

### Fermentation Broth Formulations or Cell Compositions

The present invention also relates to a fermentation broth formulation or a cell composition comprising a polypeptide of the invention. The fermentation broth product further comprises additional ingredients used in the fermentation process, for example cells (including the host cells containing the gene encoding the polypeptide of the present invention which are used to produce the polypeptide of interest), cell debris, biomass, fermentation media and/or fermentation products. In some embodiments, the composition is a cell-killed whole broth containing organic acids, killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains at least one organic acid, and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzymes. In some embodiments, insoluble components may be removed to provide a clarified liquid composition.

The whole broth formulations and cell compositions of the present invention may be produced by a method described in WO 90/15861 or WO 2010/096673.

### Compositions

The present invention also relates to compositions comprising a polypeptide of the invention, in particular cleaning compositions.

The compositions may comprise a polypeptide of the present invention as the major enzymatic component, e.g., a mono-component composition. Alternatively, the compositions may comprise multiple enzymatic activities, such as one or more enzymes selected from the group consisting of hydrolase, isomerase, ligase, lyase, oxidoreductase, or transferase, e.g., an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

The invention relates to cleaning or detergent compositions comprising a fructan degrading enzyme in combination with one or more additional cleaning composition components. The choice of additional components is within the skill of the artisan and includes conventional ingredients, including the exemplary non-limiting components set forth below.

One aspect of the invention relates to a cleaning composition comprising a polypeptide having fructan degrading activity, in particular a polypeptide comprising a GH32 domain and preferably also a GH32C domain, and at least one cleaning component.

Polypeptides having fructan degrading activity in the cleaning compositions of the invention preferably belong to the WMNE clade and comprise the motif [WPG][GMTH][NH][AEILDNMV] (SEQ ID NO: 24), preferably the motif W[GMT]N[NDEI] (SEQ ID NO: 32), and optionally also belong to the AYSN clade and comprise the motif A[YF]S[LN]D[QK](SEQ ID NO: 25) and/or the motif [HS]WGH (SEQ ID NO: 27) and/or belong to the WMNEP clade and comprise the motif [QLKEYMRVTANI] [NSYHATRD][WF][MEVKTL] [NGA] [EVDLIH][PAEHDS] [NATSKGVERQCD] (SEQ ID NO: 28).

One aspect of the invention relates to a cleaning composition comprising;
a) a polypeptide having fructan degrading activity, wherein the polypeptide is selected from the group consisting of;
   i. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 6,
   ii. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 9,
   iii. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 12,
   iv. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 15, and
   v. a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 18; and
b) at least one cleaning component, preferably selected from surfactants, builders, bleach components, polymers, dispersing agents and additional enzymes.

The fructan degrading enzyme may be included in the cleaning composition of the present invention at a level of at least 0.0001 to at least 100, at least 0.001 to at least 100, at least 0.01 to at least 100, at least 0.02 to at least 100, at least 0.01 to at least 100, at least 0.1 to at least 100, at least 0.2 to at least 100, at least 0.5 to at least 100 mg/mL, preferably, the concentration of fructan degrading enzyme in the composition is in the range 0.01 to 100, 0.1 to 50 or 1 to 10 mg/ml. Thus, the detergent composition may comprise at least 0.00008%, preferably at least 0.002%, 0.003%, 0.004%, 0.005%, 0.006%, 0.008%, 0.01%, 0.02%, 0.03%, 0.05%, 0.1%, 0.2%, 0.3%, 0.4%, 0.6%, 0.7%, 0.8%, 0.9% or 1.0% of fructan degrading enzyme protein.

### Polypeptides Having DNase Activity

A particular aspect of the invention relates to cleaning compositions as described elsewhere herein comprising, in addition to the at least one fructan degrading enzyme comprising a GH32 domain and preferably also a GH32C domain, at least one nuclease, e.g. a DNase or an RNase, and preferably an enzyme having DNase activity.

The term “DNase” means a nuclease polypeptide having DNase activity that catalyzes the hydrolytic cleavage of phosphodiester linkages in a DNA backbone, thus degrading DNA. The terms “DNase” and “a polypeptide with/having DNase activity” are used interchangeably herein. DNase activity may be determined according to the procedure described in Assay 1 or Assay 2 of PCT/EP2019/076825.

Preferably the DNase is selected from any of the enzyme classes E.C. 3.1.21.X, where X=1, 2, 3, 4, 5, 6, 7, 8 or 9, e.g. Deoxyribonuclease 1, Deoxyribonuclease IV, Type I site-specific deoxyribonuclease, Type II site-specific deoxyribonuclease, Type III site-specific deoxyribonuclease, CC-preferring endo-deoxyribonuclease, Deoxyribonuclease V, T(4) deoxyribonuclease II, T(4) deoxyribonuclease IV or E.C. 3.1.22.Y where Y=1, 2, 4 or 5, e.g. Deoxyribonuclease II, Aspergillus deoxyribonuclease K(1), Crossover junction endo-deoxyribonuclease, or Deoxyribonuclease X.

Preferably, the DNase activity is obtained from a microorganism, and the DNase is a microbial enzyme. The DNase is preferably of fungal or bacterial origin.

The DNase may be obtained from a bacterium, e.g. Bacillus, such as Bacillus licheniformis, Bacillus subtilis, Bacillus sp., Bacillus horikoshii, Bacillus horneckiae, Bacillus cibi, Bacillus idriensis, Bacillus algicola, Bacillus vietnamensis, Bacillus hwajinpoensis, Bacillus indicus, Bacillus marisflavi, Bacillus luciferensis, or Bacillus sp. SA2-6.

The DNase may also be obtained from any of the following: Pyrenochaetopsis sp., Vibrissea flavovirens, Setosphaeria rostrate, Endophragmiella valdina, Corynespora cassiicola, Paraphoma sp., Monilinia fructicola, Curvularia lunata, Penicillium reticulisporum, Penicillium quercetorum, Setophaeosphaeria sp., Alternaria, Alternaria sp., Trichoderma reesei, Chaetomium thermophilum, Scytalidium thermophilum, Metapochonia suchlasporia, Daldinia fissa, Acremonium sp., Acremonium dichromosporum, Sarocladium sp., Metarhizium sp. HNA15-2, Isaria tenuipes Scytalidium circinatum, Metarhizium lepidiotae, Thermobispora bispora, Sporormia fimetaria, Pycnidiophora cf dispera, Clavicipitaceae sp., Westerdykella sp., Humicolopsis cephalosporioides, Neosartorya massa, Roussoella intermedia, Pleosporales, Phaeosphaeria or Didymosphaeria futilis.

In some embodiments, the polypeptides having DNase activity are polypeptides comprising the PFAM domain DUF1524 ((http://pfam.xfam.org/), “The Pfam protein families database: towards a more sustainable future”, R. D. Finn, et. al. Nucleic Acids Research (2016) Database Issue 44:D279-D285”). The DUF1524 domain contains a conserved HXXP sequence (where H is the amino acid histidine, P is the amino acid proline, and X is any amino acid motif) commonly found in nucleases (M. A. Machnicka, et al. Phylogenomics and sequence-structure-function relationships in the GmrSD family of Type IV restriction enzymes, BMC Bioinformatics, 2015, 16, 336). DUF stands for domain of unknown function, and the polypeptide families comprising, e.g., DUF have been collected together in the Pfam database, which provides sequence alignments and hidden Markov models that define the collected protein domains.

Thus, in some embodiments the polypeptides having DNase activity in the composition of the invention comprise the DUF1524 domain. For further information on DNases comprising the DUF1524 domain, see WO 2017/060475, which is hereby incorporated by reference.

In some embodiments, the DNase is a NUC1 or NUC1_A DNase. A NUC1 DNase is a DNase comprising a domain termed NUC1, and polypeptides with this domain are in addition to having DNase activity characterized by comprising certain motifs. Similarly, a NUC1 sub-domain had been identified, termed the NUC1_A domain, which also is characterized by comprising certain motifs. The NUC1 and NUC1_A DNases are described in WO 2017/060475 and WO 2018/184873, which are hereby incorporated by reference.

The preparation of the polypeptide having DNase activity as described herein can e.g. be performed as described in WO 2017/059802, in particular in the sections Nucleic Acid Construct, Expression Vectors, Host Cells, Methods of Production and Fermentation Broth Formulations.

In one embodiment, the cleaning composition of the invention comprises, in addition to a fructan degrading enzyme, a polypeptide having DNase activity and a sequence identity to the polypeptide shown in SEQ ID NO: 19 of at least 60%, e.g., at least 65%, at least 70%, at least 75%, at least 80%, at least 85%, at least 90%, at least 95%, at least 96%, at least 97%, at least 98%, at least 99% or 100%. In one embodiment the polypeptide has an amino acid sequence that comprises or consists of SEQ ID NO: 19. The polypeptide may also be a fragment of SEQ ID NO: 19 which has DNase activity.

In one embodiment, the cleaning composition of the invention comprises, in addition to a fructan degrading enzyme, a polypeptide having DNase activity and a sequence identity to the polypeptide shown in SEQ ID NO: 20 of at least 60%, e.g., at least 65%, at least 70%, at least 75%, at least 80%, at least 85%, at least 90%, at least 95%, at least 96%, at least 97%, at least 98%, at least 99% or 100%. In one embodiment the DNase has an amino acid sequence that comprises or consists of SEQ ID NO: 20. The polypeptide may also be a fragment of SEQ ID NO: 20 which has DNase activity.

In one embodiment, the cleaning composition of the invention comprises, in addition to a fructan degrading enzyme, a polypeptide having DNase activity and a sequence identity to the polypeptide shown in SEQ ID NO: 21 of at least 60%, e.g., at least 65%, at least 70%, at least 75%, at least 80%, at least 85%, at least 90%, at least 95%, at least 96%, at least 97%, at least 98%, at least 99% or 100%. In one embodiment the DNase has an amino acid sequence that comprises or consists of SEQ ID NO: 21. The polypeptide may also be a fragment of SEQ ID NO: 21 which has DNase activity.

In one embodiment, the cleaning composition of the invention comprises, in addition to a fructan degrading enzyme, a polypeptide having DNase activity and a sequence identity to the polypeptide shown in SEQ ID NO: 22 of at least 60%, e.g., at least 65%, at least 70%, at least 75%, at least 80%, at least 85%, at least 90%, at least 95%, at least 96%, at least 97%, at least 98%, at least 99% or 100%. In one embodiment the DNase has an amino acid sequence that comprises or consists of SEQ ID NO: 22.

The polypeptide having DNase activity may also be a fragment of SEQ ID NO: 22 which has DNase activity. The polypeptide of SEQ ID NO: 22 may be expressed with different N-terminal truncations, for example as a 206 amino acid residue polypeptide corresponding to amino acids 16 to 221 of SEQ ID NO: 22, or a 204 amino acid residue polypeptide corresponding to amino acids 18 to 221 of SEQ ID NO: 22, or a mixture of two of more of such polypeptides.

In one embodiment, the cleaning composition of the invention comprises, in addition to a fructan degrading enzyme, a polypeptide having DNase activity and a sequence identity to the polypeptide shown in SEQ ID NO: 23 of at least 60%, e.g., at least 65%, at least 70%, at least 75%, at least 80%, at least 85%, at least 90%, at least 95%, at least 96%, at least 97%, at least 98%, at least 99% or 100%. In one embodiment the DNase has an amino acid sequence that comprises or consists of SEQ ID NO: 23. The polypeptide may also be a fragment of SEQ ID NO: 23 which has DNase activity.

In one particular embodiment, the cleaning composition comprises:
a fructan degrading enzyme that comprises or consists of SEQ ID NO: 3, or which is a variant thereof having at least 80%, at least 85%, at least 90%, at least 95%, at least 96%, at least 97%, at least 98% or at least 99% sequence identity to SEQ ID NO: 3; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 19, or which is a variant thereof having at least 80%, at least 85%, at least 90%, at least 95%, at least 96%, at least 97%, at least 98% or at least 99% sequence identity to SEQ ID NO: 19.

In another particular embodiment, the cleaning composition comprises:
a fructan degrading enzyme that comprises or consists of SEQ ID NO: 3, or which is a variant thereof having at least 80%, at least 85%, at least 90%, at least 95%, at least 96%, at least 97%, at least 98% or at least 99% sequence identity to SEQ ID NO: 3; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 20, or which is a variant thereof having at least 80%, at least 85%, at least 90%, at least 95%, at least 96%, at least 97%, at least 98% or at least 99% sequence identity to SEQ ID NO: 20.

In another particular embodiment, the cleaning composition comprises:
a fructan degrading enzyme that comprises or consists of SEQ ID NO: 3, or which is a variant thereof having at least 80%, at least 85%, at least 90%, at least 95%, at least 96%, at least 97%, at least 98% or at least 99% sequence identity to SEQ ID NO: 3; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 21, or which is a variant thereof having at least 80%, at least 85%, at least 90%, at least 95%, at least 96%, at least 97%, at least 98% or at least 99% sequence identity to SEQ ID NO: 21.

In another particular embodiment, the cleaning composition comprises:
a fructan degrading enzyme that comprises or consists of SEQ ID NO: 3, or which is a variant thereof having at least 80%, at least 85%, at least 90%, at least 95%, at least 96%, at least 97%, at least 98% or at least 99% sequence identity to SEQ ID NO: 3; and preferably comprising the motif of SEQ ID NO: 24 more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 22, or which is a variant thereof having at least 80%, at least 85%, at least 90%, at least 95%, at least 96%, at least 97%, at least 98% or at least 99% sequence identity to SEQ ID NO: 22.

In another particular embodiment, the cleaning composition comprises:
a fructan degrading enzyme that comprises or consists of SEQ ID NO: 3, or which is a variant thereof having at least 80%, at least 85%, at least 90%, at least 95%, at least 96%, at least 97%, at least 98% or at least 99% sequence identity to SEQ ID NO: 3; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 23, or which is a variant thereof having at least 80%, at least 85%, at least 90%, at least 95%, at least 96%, at least 97%, at least 98% or at least 99% sequence identity to SEQ ID NO: 23.

In another particular embodiment, the cleaning composition comprises:
a fructan degrading enzyme that comprises or consists of SEQ ID NO: 6, or which is a variant thereof having at least 80%, at least 85%, at least 90%, at least 95%, at least 96%, at least 97%, at least 98% or at least 99% sequence identity to SEQ ID NO: 6; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 19, or which is a variant thereof having at least 80%, at least 85%, at least 90%, at least 95%, at least 96%, at least 97%, at least 98% or at least 99% sequence identity to SEQ ID NO: 19.

In another particular embodiment, the cleaning composition comprises:
a fructan degrading enzyme that comprises or consists of SEQ ID NO: 6, or which is a variant thereof having at least 80%, at least 85%, at least 90%, at least 95%, at least 96%, at least 97%, at least 98% or at least 99% sequence identity to SEQ ID NO: 6; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 20, or which is a variant thereof having at least 80%, at least 85%, at least 90%, at least 95%, at least 96%, at least 97%, at least 98% or at least 99% sequence identity to SEQ ID NO: 20.

In another particular embodiment, the cleaning composition comprises:
a fructan degrading enzyme that comprises or consists of SEQ ID NO: 6, or which is a variant thereof having at least 80%, at least 85%, at least 90%, at least 95%, at least 96%, at least 97%, at least 98% or at least 99% sequence identity to SEQ ID NO: 6; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 21, or which is a variant thereof having at least 80%, at least 85%, at least 90%, at least 95%, at least 96%, at least 97%, at least 98% or at least 99% sequence identity to SEQ ID NO: 21.

In another particular embodiment, the cleaning composition comprises:
a fructan degrading enzyme that comprises or consists of SEQ ID NO: 6, or which is a variant thereof having at least 80%, at least 85%, at least 90%, at least 95%, at least 96%, at least 97%, at least 98% or at least 99% sequence identity to SEQ ID NO: 6; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 22, or which is a variant thereof having at least 80%, at least 85%, at least 90%, at least 95%, at least 96%, at least 97%, at least 98% or at least 99% sequence identity to SEQ ID NO: 22.

In another particular embodiment, the cleaning composition comprises:
a fructan degrading enzyme that comprises or consists of SEQ ID NO: 6, or which is a variant thereof having at least 80%, at least 85%, at least 90%, at least 95%, at least 96%, at least 97%, at least 98% or at least 99% sequence identity to SEQ ID NO: 6; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 23, or which is a variant thereof having at least 80%, at least 85%, at least 90%, at least 95%, at least 96%, at least 97%, at least 98% or at least 99% sequence identity to SEQ ID NO: 23.

In one particular embodiment, the cleaning composition comprises:
a fructan degrading enzyme that comprises or consists of SEQ ID NO: 9, or which is a variant thereof having at least 80%, at least 85%, at least 90%, at least 95%, at least 96%, at least 97%, at least 98% or at least 99% sequence identity to SEQ ID NO: 9; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 19, or which is a variant thereof having at least 80%, at least 85%, at least 90%, at least 95%, at least 96%, at least 97%, at least 98% or at least 99% sequence identity to SEQ ID NO: 19.

In another particular embodiment, the cleaning composition comprises:
a fructan degrading enzyme that comprises or consists of SEQ ID NO: 9, or which is a variant thereof having at least 80%, at least 85%, at least 90%, at least 95%, at least 96%, at least 97%, at least 98% or at least 99% sequence identity to SEQ ID NO: 9; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 20, or which is a variant thereof having at least 80%, at least 85%, at least 90%, at least 95%, at least 96%, at least 97%, at least 98% or at least 99% sequence identity to SEQ ID NO: 20.

In another particular embodiment, the cleaning composition comprises:
a fructan degrading enzyme that comprises or consists of SEQ ID NO: 9, or which is a variant thereof having at least 80%, at least 85%, at least 90%, at least 95%, at least 96%, at least 97%, at least 98% or at least 99% sequence identity to SEQ ID NO: 9; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 21, or which is a variant thereof having at least 80%, at least 85%, at least 90%, at least 95%, at least 96%, at least 97%, at least 98% or at least 99% sequence identity to SEQ ID NO: 21.

In another particular embodiment, the cleaning composition comprises:
a fructan degrading enzyme that comprises or consists of SEQ ID NO: 9, or which is a variant thereof having at least 80%, at least 85%, at least 90%, at least 95%, at least 96%, at least 97%, at least 98% or at least 99% sequence identity to SEQ ID NO: 9; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 22, or which is a variant thereof having at least 80%, at least 85%, at least 90%, at least 95%, at least 96%, at least 97%, at least 98% or at least 99% sequence identity to SEQ ID NO: 22.

In another particular embodiment, the cleaning composition comprises:
a fructan degrading enzyme that comprises or consists of SEQ ID NO: 9, or which is a variant thereof having at least 80%, at least 85%, at least 90%, at least 95%, at least 96%, at least 97%, at least 98% or at least 99% sequence identity to SEQ ID NO: 9; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 23, or which is a variant thereof having at least 80%, at least 85%, at least 90%, at least 95%, at least 96%, at least 97%, at least 98% or at least 99% sequence identity to SEQ ID NO: 23.

In one particular embodiment, the cleaning composition comprises:
a fructan degrading enzyme that comprises or consists of SEQ ID NO: 12, or which is a variant thereof having at least 80%, at least 85%, at least 90%, at least 95%, at least 96%, at least 97%, at least 98% or at least 99% sequence identity to SEQ ID NO: 12; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 19, or which is a variant thereof having at least 80%, at least 85%, at least 90%, at least 95%, at least 96%, at least 97%, at least 98% or at least 99% sequence identity to SEQ ID NO: 19.

In another particular embodiment, the cleaning composition comprises:
a fructan degrading enzyme that comprises or consists of SEQ ID NO: 12, or which is a variant thereof having at least 80%, at least 85%, at least 90%, at least 95%, at least 96%, at least 97%, at least 98% or at least 99% sequence identity to SEQ ID NO: 12; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 20, or which is a variant thereof having at least 80%, at least 85%, at least 90%, at least 95%, at least 96%, at least 97%, at least 98% or at least 99% sequence identity to SEQ ID NO: 20.

In another particular embodiment, the cleaning composition comprises:
a fructan degrading enzyme that comprises or consists of SEQ ID NO: 12, or which is a variant thereof having at least 80%, at least 85%, at least 90%, at least 95%, at least 96%, at least 97%, at least 98% or at least 99% sequence identity to SEQ ID NO: 12; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 21, or which is a variant thereof having at least 80%, at least 85%, at least 90%, at least 95%, at least 96%, at least 97%, at least 98% or at least 99% sequence identity to SEQ ID NO: 21.

In another particular embodiment, the cleaning composition comprises:
a fructan degrading enzyme that comprises or consists of SEQ ID NO: 12, or which is a variant thereof having at least 80%, at least 85%, at least 90%, at least 95%, at least 96%, at least 97%, at least 98% or at least 99% sequence identity to SEQ ID NO: 12; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 22, or which is a variant thereof having at least 80%, at least 85%, at least 90%, at least 95%, at least 96%, at least 97%, at least 98% or at least 99% sequence identity to SEQ ID NO: 22.

In another particular embodiment, the cleaning composition comprises:
a fructan degrading enzyme that comprises or consists of SEQ ID NO: 12, or which is a variant thereof having at least 80%, at least 85%, at least 90%, at least 95%, at least 96%, at least 97%, at least 98% or at least 99% sequence identity to SEQ ID NO: 12; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 23, or which is a variant thereof having at least 80%, at least 85%, at least 90%, at least 95%, at least 96%, at least 97%, at least 98% or at least 99% sequence identity to SEQ ID NO: 23.

In one particular embodiment, the cleaning composition comprises:
a fructan degrading enzyme that comprises or consists of SEQ ID NO: 15, or which is a variant thereof having at least 80%, at least 85%, at least 90%, at least 95%, at least 96%, at least 97%, at least 98% or at least 99% sequence identity to SEQ ID NO: 15; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 19, or which is a variant thereof having at least 80%, at least 85%, at least 90%, at least 95%, at least 96%, at least 97%, at least 98% or at least 99% sequence identity to SEQ ID NO: 19.

In another particular embodiment, the cleaning composition comprises:
a fructan degrading enzyme that comprises or consists of SEQ ID NO: 15, or which is a variant thereof having at least 80%, at least 85%, at least 90%, at least 95%, at least 96%, at least 97%, at least 98% or at least 99% sequence identity to SEQ ID NO: 15; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 20, or which is a variant thereof having at least 80%, at least 85%, at least 90%, at least 95%, at least 96%, at least 97%, at least 98% or at least 99% sequence identity to SEQ ID NO: 20.

In another particular embodiment, the cleaning composition comprises:
a fructan degrading enzyme that comprises or consists of SEQ ID NO: 15, or which is a variant thereof having at least 80%, at least 85%, at least 90%, at least 95%, at least 96%, at least 97%, at least 98% or at least 99% sequence identity to SEQ ID NO: 15; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 21, or which is a variant thereof having at least 80%, at least 85%, at least 90%, at least 95%, at least 96%, at least 97%, at least 98% or at least 99% sequence identity to SEQ ID NO: 21.

In another particular embodiment, the cleaning composition comprises:
a fructan degrading enzyme that comprises or consists of SEQ ID NO: 15, or which is a variant thereof having at least 80%, at least 85%, at least 90%, at least 95%, at least 96%, at least 97%, at least 98% or at least 99% sequence identity to SEQ ID NO: 15; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 22, or which is a variant thereof having at least 80%, at least 85%, at least 90%, at least 95%, at least 96%, at least 97%, at least 98% or at least 99% sequence identity to SEQ ID NO: 22.

In another particular embodiment, the cleaning composition comprises:
a fructan degrading enzyme that comprises or consists of SEQ ID NO: 15, or which is a variant thereof having at least 80%, at least 85%, at least 90%, at least 95%, at least 96%, at least 97%, at least 98% or at least 99% sequence identity to SEQ ID NO: 15; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 23, or which is a variant thereof having at least 80%, at least 85%, at least 90%, at least 95%, at least 96%, at least 97%, at least 98% or at least 99% sequence identity to SEQ ID NO: 23.

In one particular embodiment, the cleaning composition comprises:
a fructan degrading enzyme that comprises or consists of SEQ ID NO: 18, or which is a variant thereof having at least 80%, at least 85%, at least 90%, at least 95%, at least 96%, at least 97%, at least 98% or at least 99% sequence identity to SEQ ID NO: 18; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 19, or which is a variant thereof having at least 80%, at least 85%, at least 90%, at least 95%, at least 96%, at least 97%, at least 98% or at least 99% sequence identity to SEQ ID NO: 19.

In another particular embodiment, the cleaning composition comprises:
a fructan degrading enzyme that comprises or consists of SEQ ID NO: 18, or which is a variant thereof having at least 80%, at least 85%, at least 90%, at least 95%, at least 96%, at least 97%, at least 98% or at least 99% sequence identity to SEQ ID NO: 18; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 20, or which is a variant thereof having at least 80%, at least 85%, at least 90%, at least 95%, at least 96%, at least 97%, at least 98% or at least 99% sequence identity to SEQ ID NO: 20.

In another particular embodiment, the cleaning composition comprises:
a fructan degrading enzyme that comprises or consists of SEQ ID NO: 18, or which is a variant thereof having at least 80%, at least 85%, at least 90%, at least 95%, at least 96%, at least 97%, at least 98% or at least 99% sequence identity to SEQ ID NO: 18; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 21, or which is a variant thereof having at least 80%, at least 85%, at least 90%, at least 95%, at least 96%, at least 97%, at least 98% or at least 99% sequence identity to SEQ ID NO: 21.

In another particular embodiment, the cleaning composition comprises:
a fructan degrading enzyme that comprises or consists of SEQ ID NO: 18, or which is a variant thereof having at least 80%, at least 85%, at least 90%, at least 95%, at least 96%, at least 97%, at least 98% or at least 99% sequence identity to SEQ ID NO: 18; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 22, or which is a variant thereof having at least 80%, at least 85%, at least 90%, at least 95%, at least 96%, at least 97%, at least 98% or at least 99% sequence identity to SEQ ID NO: 22.

In another particular embodiment, the cleaning composition comprises:
a fructan degrading enzyme that comprises or consists of SEQ ID NO: 18, or which is a variant thereof having at least 80%, at least 85%, at least 90%, at least 95%, at least 96%, at least 97%, at least 98% or at least 99% sequence identity to SEQ ID NO: 18; and preferably comprising the motif of SEQ ID NO: 24, more preferably the motif of SEQ ID NO: 32, and optionally also the motif of SEQ ID NO: 25, SEQ ID NO: 27 and/or SEQ ID NO: 28; and
DNase that comprises or consists of SEQ ID NO: 23, or which is a variant thereof having at least 80%, at least 85%, at least 90%, at least 95%, at least 96%, at least 97%, at least 98% or at least 99% sequence identity to SEQ ID NO: 23.

In another embodiment, the cleaning composition of the invention may comprise any of the DNase polypeptides disclosed in WO 2015/155350, WO 2017/059802, WO 2017/060475, WO 2017/060505, WO 2017/064269, WO 2018/011277, WO 2018/177938, WO 2018/185285, WO 2018/184873, WO 2019/081721 or WO 2019/081724, the contents of which are incorporated herein by reference.

When present in a cleaning composition of the invention, the DNase polypeptide will typically be present in an amount of from 0.01 to 1000 ppm, from 0.1 to 1000 ppm, from 1 ppm to 1000 ppm, from 10 ppm to 1000 ppm, from 50 ppm to 1000 ppm, from 100 ppm to 1000 ppm, from 150 ppm to 1000 ppm, from 200 ppm to 1000 ppm, from 250 ppm to 1000 ppm, from 250 ppm to 750 ppm, or from 250 ppm to 500 ppm, based on active protein.

### Cleaning Components

The choice of cleaning components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

### Surfactants

The cleaning composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant is typically present at a level of from about 1% to 70% by weight, such as about 1 wt % to about 40 wt %, or about 3 wt % to about 20 wt %, or about 3 wt % to about 10 wt %.

The one or more surfactants are chosen based on the desired cleaning application, and may include any conventional surfactants known in the art.

When included therein the detergent will usually contain from about 1% to about 70% by weight of an anionic surfactant, such as from about 5 wt % to about 50 wt %, including from about 5 wt % to about 20 wt %, or from about 15 wt % to about 20 wt %, or from about 20 wt % to about 25 wt % or at least 30 wt %, at least 40 wt % or at least 50 wt % of an anionic surfactant. Non-limiting examples of anionic surfactants include sulfates and sulfonates, in particular, alkylbenzenesulfonates, such as linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.

When included therein the detergent will usually contain from about 1% to about 40% by weigh of a cationic surfactant, for example from about 0.5% to about 30%, in particular from about 1% to about 20%, from about 3% to about 10%, such as from about 3% to about 5%, from about 8% to about 12% or from about 10% to about 12%. Non-limiting examples of cationic surfactants include alkyldimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, ester quats, and combinations thereof.

When included therein the detergent will usually contain from about 0.2% to about 40% by weight of a nonionic surfactant, for example from about 0.5 wt % to about 30 wt %, in particular from about 1 wt % to about 20 wt %, from about 3 wt % to about 10 wt %, such as from about 3% wt to about 5 wt %, from about 8 wt % to about 12 wt %, or from about 10 wt % to about 12 wt %. Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

When included therein the detergent will usually contain from about 0.01% to about 10% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, and combinations thereof.

When included therein the detergent will usually contain from about 0.01% to about 10% by weight of a zwitterionic surfactant. Non-limiting examples of zwitterionic surfactants include betaines such as alkyldimethylbetaines, sulfobetaines, and combinations thereof.

Typically, more than one surfactant is present in the cleaning composition, for example at least one anionic and at least one non-ionic surfactant. Preferably, the amount of all surfactant present (total amount) i.e. the amount of anionic, non-ionic, zwitterionic and cationic surfactant present is preferably from about 1 wt % to 80 wt % by weight, such as about 1 wt % to 70 wt %, such as about 1 wt % to 50 wt % such as about 1 wt % to about 40 wt %, or about 5 wt % to about 40 wt %, or about 10 wt % to about 60 wt %. The ratio between the surfactants present depends on the specific composition but the weight ratios may be when an anionic and non-ionic surfactant is included in the composition a weight ratio of the anionic to nonionic surfactant from; 30:1 to 10:1, 20:1 to 1:10, 25:1 to 1:2, 20:1 to 1:5.

One embodiment relates to a cleaning composition comprising a fructan degrading enzyme, wherein the cleaning component is at least one surfactant, preferably anionic and/or nonionic, preferably wherein the composition comprises from 1 to 70 wt %, preferably from 5 to 40 wt % surfactant, wherein the surfactant preferably is selected from alkylbenzenesulfonates e.g. LAS, alkyl sulfates (AS) and mixtures thereof, preferably the cleaning composition comprises at least 20 wt % alkylbenzenesulfonate surfactant.

One embodiment relates to a cleaning composition comprising a fructan degrading enzyme, wherein the cleaning composition comprises at least one anionic surfactant and wherein the cleaning composition additionally comprises a nonionic surfactant, and preferably wherein the weight ratio of the anionic to nonionic surfactant is from 25:1 to 1:2 or from 1.5:1 to 1:10.

### Builders and Co-Builders

The cleaning composition may contain about 0-65% by weight, such as about 5% to about 50%, such as about 0.5% to about 20% of a detergent builder or co-builder, or a mixture thereof. In a dish wash detergent, the level of builder is typically 40-65%, particularly 50-65%. The builder and/or co-builder may particularly be a chelating agent that forms water-soluble complexes with Ca and Mg. Any builder and/or co-builder known in the art for use in cleaning detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl)inulin (CMI), and combinations thereof.

The detergent composition may also contain 0-50% by weight, such as about 5% to about 30%, of a detergent co-builder. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis-(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethyl)ethylenediamine-N,N′,N″-triacetic acid (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053

### Bleaching Systems

The cleaning composition may contain 0-30% by weight, such as about 1% to about 20%, such as about 0.01% to about 10% of a bleaching system. Any bleaching system comprising components known in the art for use in cleaning detergents may be utilized. Suitable bleaching system components include sources of hydrogen peroxide; sources of peracids; and bleach catalysts or boosters.

Suitable sources of hydrogen peroxide are inorganic persalts, including alkali metal salts such as sodium percarbonate and sodium perborates (usually mono- or tetrahydrate), and hydrogen peroxide—urea (1/1).

Peracids may be (a) incorporated directly as preformed peracids or (b) formed in situ in the wash liquor from hydrogen peroxide and a bleach activator (perhydrolysis) or (c) formed in situ in the wash liquor from hydrogen peroxide and a perhydrolase and a suitable substrate for the latter, e.g., an ester.

Suitable preformed peracids include, but are not limited to, peroxycarboxylic acids such as peroxybenzoic acid and its ring-substituted derivatives, peroxy-α-naphthoic acid, peroxyphthalic acid, peroxylauric acid, peroxystearic acid, ε-phthalimidoperoxycaproic acid [phthalimidoperoxyhexanoic acid (PAP)], and o-carboxybenzamidoperoxycaproic acid; aliphatic and aromatic diperoxydicarboxylic acids such as diperoxydodecanedioic acid, diperoxyazelaic acid, diperoxysebacic acid, diperoxybrassylic acid, 2-decyldiperoxybutanedioic acid, and diperoxyphthalic, -isophthalic and -terephthalic acids; perimidic acids; peroxymonosulfuric acid; peroxydisulfuric acid; peroxyphosphoric acid; peroxysilicic acid; and mixtures of said compounds. It is understood that the peracids mentioned may in some cases be best added as suitable salts, such as alkali metal salts (e.g., Oxone®) or alkaline earth-metal salts.

Suitable bleach activators include those belonging to the class of esters, amides, imides, nitriles or anhydrides and, where applicable, salts thereof. Suitable examples are tetraacetylethylenediamine (TAED), sodium 4-[(3,5,5-trimethylhexanoyl)oxy]benzene-1-sulfonate (ISONOBS), sodium 4-(dodecanoyloxy)benzene-1-sulfonate (LOBS), sodium 4-(decanoyloxy)benzene-1-sulfonate, 4-(decanoyloxy)benzoic acid (DOBA), sodium 4-(nonanoyloxy)benzene-1-sulfonate (NOBS), and/or those disclosed in WO98/17767. A particular family of bleach activators of interest was disclosed in EP624154 and particularly preferred in that family is acetyl triethyl citrate (ATC). ATC or a short chain triglyceride like triacetin has the advantage that they are environmentally friendly. Furthermore, acetyl triethyl citrate and triacetin have good hydrolytical stability in the product upon storage and are efficient bleach activators. Finally, ATC is multifunctional, as the citrate released in the perhydrolysis reaction may function as a builder.

### Bleach Catalysts and Boosters

The bleaching system may also include a bleach catalyst or booster. Some non-limiting examples of bleach catalysts that may be used in the compositions of the present invention include manganese oxalate, manganese acetate, manganese-collagen, cobalt-amine catalysts and manganese triazacyclononane (MnTACN) catalysts; particularly preferred are complexes of manganese with 1,4,7-trimethyl-1,4,7-triazacyclononane (Me3-TACN) or 1,2,4,7-tetramethyl-1,4,7-triazacyclononane (Me4-TACN), in particular Me3-TACN, such as the dinuclear manganese complex [(Me3-TACN)Mn(O)3Mn(Me3-TACN)](PF6)2, and [2,2′,2″-nitrilotris(ethane-1,2-diylazanylylidene-κN-methanylylidene)triphenolato-κ3O]manganese(III). The bleach catalysts may also be other metal compounds; such as iron or cobalt complexes.

In some embodiments, where a source of a peracid is included, an organic bleach catalyst or bleach booster may be used having one of the following formulae:

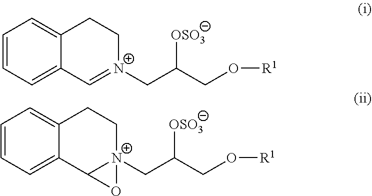

(iii) and mixtures thereof; wherein each R1 is independently a branched alkyl group containing from 9 to 24 carbons or linear alkyl group containing from 11 to 24 carbons, preferably each R1 is independently a branched alkyl group containing from 9 to 18 carbons or linear alkyl group containing from 11 to 18 carbons, more preferably each R1 is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl.

Other exemplary bleaching systems are described e.g. in WO2007/087258, WO2007/087244, WO2007/087259, EP1867708 (Vitamin K) and WO2007/087242. Suitable photobleaches may for example be sulfonated zinc or aluminium phthalocyanines.

### Metal Care Agents

Metal care agents may prevent or reduce the tarnishing, corrosion or oxidation of metals, including aluminium, stainless steel and non-ferrous metals, such as silver and copper. Suitable examples include one or more of the following:

(a) benzotriazoles, including benzotriazole or bis-benzotriazole and substituted derivatives thereof. Benzotriazole derivatives are those compounds in which the available substitution sites on the aromatic ring are partially or completely substituted. Suitable substituents include linear or branch-chain Ci-C20-alkyl groups (e.g., C1-C20-alkyl groups) and hydroxyl, thio, phenyl or halogen such as fluorine, chlorine, bromine and iodine.

(b) metal salts and complexes chosen from the group consisting of zinc, manganese, titanium, zirconium, hafnium, vanadium, cobalt, gallium and cerium salts and/or complexes, the metals being in one of the oxidation states II, III, IV, V or VI. In one aspect, suitable metal salts and/or metal complexes may be chosen from the group consisting of Mn(II) sulphate, Mn(II) citrate, Mn(II) stearate, Mn(II) acetylacetonate, K\hat{ }TiF6 (e.g., K2TiF6), K\hat{ }ZrF6 (e.g., K2ZrF6), CoSO4, Co(NOs)2 and Ce(NOs)3, zinc salts, for example zinc sulphate, hydrozincite or zinc acetate;

(c) silicates, including sodium or potassium silicate, sodium disilicate, sodium metasilicate, crystalline phyllosilicate and mixtures thereof.

Further suitable organic and inorganic redox-active substances that act as silver/copper corrosion inhibitors are disclosed in WO 94/26860 and WO 94/26859. Preferably the composition of the invention comprises from 0.1 to 5% by weight of the composition of a metal care agent, preferably the metal care agent is a zinc salt.

### Hydrotropes

The cleaning composition may contain 0-10% by weight, for example 0-5% by weight, such as about 0.5 to about 5%, or about 3% to about 5%, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Polymers

The cleaning composition may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Suitable examples include PVP-K15, PVP-K30, ChromaBond S-400, ChromaBond S-403E and Chromabond S-100 from Ashland Aqualon, and Sokalan® HP 165, Sokalan® HP 50 (Dispersing agent), Sokalan® HP 53 (Dispersing agent), Sokalan® HP 59 (Dispersing agent), Sokalan® HP 56 (dye transfer inhibitor), Sokalan® HP 66 K (dye transfer inhibitor) from BASF. Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated. Particularly preferred polymer is ethoxylated homopolymer Sokalan® HP 20 from BASF, which helps to prevent redeposition of soil in the wash liquor.

### Fabric Hueing Agents

The cleaning compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO2007/087243.

### Dispersants

The cleaning compositions of the present invention can also contain dispersants. In particular, powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

### Dye Transfer Inhibiting Agents

The cleaning compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

### Fluorescent Whitening Agent

The cleaning compositions of the present invention will preferably also contain additional components that may tint articles being cleaned, such as fluorescent whitening agents or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2,2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]-benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins. Suitable fluorescent brightener levels include lower levels of from about 0.01, from 0.05, from about 0.1 or even from about 0.2 wt % to upper levels of 0.5 or even 0.75 wt %.

### Soil Release Polymers

The cleaning compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalate based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers is amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore, random graft co-polymers are suitable soil release polymers. Suitable graft co-polymers are described in more detail in WO 2007/138054, WO 2006/108856 and WO 2006/113314 (hereby incorporated by reference). Suitable polyethylene glycol polymers include random graft co-polymers comprising: (i) hydrophilic backbone comprising polyethylene glycol; and (ii) side chain(s) selected from the group consisting of: C4-C25 alkyl group, polypropylene, polybutylene, vinyl ester of a saturated C1-C6 mono-carboxylic acid, CI-C6 alkyl ester of acrylic or methacrylic acid, and mixtures thereof. Suitable polyethylene glycol polymers have a polyethylene glycol backbone with random grafted polyvinyl acetate side chains. The average molecular weight of the polyethylene glycol backbone can be in the range of from 2,000 Da to 20,000 Da, or from 4,000 Da to 8,000 Da. The molecular weight ratio of the polyethylene glycol backbone to the polyvinyl acetate side chains can be in the range of from 1:1 to 1:5, or from 1:1.2 to 1:2. The average number of graft sites per ethylene oxide units can be less than 1, or less than 0.8, the average number of graft sites per ethylene oxide units can be in the range of from 0.5 to 0.9, or the average number of graft sites per ethylene oxide units can be in the range of from 0.1 to 0.5, or from 0.2 to 0.4. A suitable polyethylene glycol polymer is Sokalan HP22. Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

### Anti-Redeposition Agents

The cleaning compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

### Rheology Modifiers

The cleaning compositions of the present invention may also include one or more rheology modifiers, structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040.

Other suitable cleaning composition components include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Polymers

The cleaning composition may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Suitable examples include PVP-K15, PVP-K30, ChromaBond S-400, ChromaBond S-403E and Chromabond S-100 from Ashland Aqualon, and Sokalan® HP 165, Sokalan® HP 50 (Dispersing agent), Sokalan® HP 53 (Dispersing agent), Sokalan® HP 59 (Dispersing agent), Sokalan® HP 56 (dye transfer inhibitor), Sokalan® HP 66 K (dye transfer inhibitor) from BASF. Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated. Particularly preferred polymer is ethoxylated homopolymer Sokalan® HP 20 from BASF, which helps to prevent redeposition of soil in the wash liquor.

### Additional Enzymes

The cleaning composition may comprise, in addition to the at least one fructan degrading enzyme and optionally at least one DNase, one or more additional enzymes such as one or more lipase, cutinase, an amylase, carbohydrase, cellulase, pectinase, mannanase, arabinase, galactanase, xylanase, oxidase, e.g., a laccase, and/or peroxidase. In general, the properties of the selected enzymes should be compatible with the selected detergent, (i.e., pH-optimum, compatibility with other enzymatic and non-enzymatic ingredients, etc.), and the enzymes should be present in effective amounts.

### Mannanases

Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 1999/064619. A commercially available mannanase is Mannaway (Novozymes A/S).

### Cellulases

Suitable cellulases include complete cellulases or mono-component endoglucanases of bacterial or fungal origin. Chemically or genetically modified mutants are included. The cellulase may for example be a mono-component or a mixture of mono-component endo-1,4-beta-glucanase often just termed endoglucanases. Suitable cellulases include a fungal cellulase from Humicola insolens (U.S. Pat. No. 4,435,307) or from Trichoderma, e.g. T. reesei or T. viride. Examples of cellulases are described in EP 0 495 257. Other suitable cellulases are from Thielavia e.g. Thielavia terrestris as described in WO 96/29397 or Fusarium oxysporum as described in WO 91/17244 or from Bacillus as described in, WO 02/099091 and JP 2000210081. Other examples are cellulase variants such as those described in WO 94/07998, EP 0 531 315, U.S. Pat. Nos. 5,457,046, 5,686,593, 5,763,254, WO 95/24471, WO 98/12307 Commercially available cellulases include Carezyme®, Celluzyme®, Celluclean®, Celluclast® and Endolase®; Renozyme®; Whitezyme® (Novozymes A/S) Puradax®, Puradax HA, and Puradax EG (available from Genencor).

### Proteases

Suitable proteases may be of any origin, but are preferably of bacterial or fungal origin, optionally in the form of protein engineered or chemically modified mutants. The protease may be an alkaline protease, such as a serine protease or a metalloprotease. A serine protease may for example be of the S1 family, such as trypsin, or the S8 family such as a subtilisin. A metalloprotease may for example be a thermolysin, e.g. from the M4 family, or another metalloprotease such as those from the M5, M7 or M35 families.

The term “subtilases” refers to a sub-group of serine proteases according to Siezen et al., Protein Eng. 4 (1991) 719-737 and Siezen et al., Protein Sci. 6 (1997) 501-523. Serine proteases are a subgroup of proteases characterized by having a serine in the active site, which forms a covalent adduct with the substrate. The subtilases may be divided into six subdivisions, the Subtilisin family, the Thermitase family, the Proteinase K family, the Lantibiotic peptidase family, the Kexin family and the Pyrolysin family.

Although proteases suitable for detergent use may be obtained from a variety of organisms, including fungi such as Aspergillus, detergent proteases have generally been obtained from bacteria and in particular from Bacillus. Examples of Bacillus species from which subtilases have been derived include Bacillus lentus, Bacillus alkalophilus, Bacillus subtilis, Bacillus amyloliquefaciens, Bacillus licheniformis, Bacillus pumilus and Bacillus gibsonii. Particular subtilisins include subtilisin lentus, subtilisin Novo, subtilisin Carlsberg, subtilisin BPN′, subtilisin 309, subtilisin 147 and subtilisin 168 and e.g. protease PD138 (described in WO 93/18140). Other useful proteases are e.g. those described in WO 01/16285 and WO 02/16547.

Examples of trypsin-like proteases include the Fusarium protease described in WO 94/25583 and WO 2005/040372, and the chymotrypsin proteases derived from Cellumonas described in WO 2005/052161 and WO 2005/052146.

Examples of metalloproteases include the neutral metalloproteases described in WO 2007/044993 such as those derived from Bacillus amyloliquefaciens, as well as e.g. the metalloproteases described in WO 2015/158723 and WO 2016/075078.

Examples of useful proteases are the protease variants described in WO 89/06279 WO 92/19729, WO 96/34946, WO 98/20115, WO 98/20116, WO 99/11768, WO 01/44452, WO 03/006602, WO 2004/003186, WO 2004/041979, WO 2007/006305, WO 2011/036263, WO 2014/207227, WO 2016/087617 and WO 2016/174234. Preferred protease variants may, for example, comprise one or more of the mutations selected from the group consisting of: S3T, V4I, S9R, S9E, A15T, S24G, S24R, K27R, N42R, S55P, G59E, G59D, N60D, N60E, V66A, N74D, S85R, A96S, S97G, S97D, S97A, S97SD, S99E, S99D, S99G, S99M, S99N, S99R, S99H, S101A, V1021, V102Y, V102N, S104A, G116V, G116R, H118D, H118N, A120S, S126L, P127Q, S128A, S154D, A156E, G157D, G157P, S158E, Y161A, R164S, Q176E, N179E, S182E, Q185N, A188P, G189E, V193M, N198D, V1991, Q200L, Y203W, S206G, L211Q, L211D, N212D, N212S, M216S, A226V, K229L, Q230H, Q239R, N246K, S253D, N255W, N255D, N255E, L256E, L256D T268A and R269H, wherein position numbers correspond to positions of the Bacillus lentus protease shown in SEQ ID NO: 1 of WO 2016/001449. Protease variants having one or more of these mutations are preferably variants of the Bacillus lentus protease (Savinase®, also known as subtilisin 309) shown in SEQ ID NO: 1 of WO 2016/001449 or of the Bacillus amyloliquefaciens protease (BPN′) shown in SEQ ID NO: 2 of WO 2016/001449. Such protease variants preferably have at least 80% sequence identity to SEQ ID NO: 1 or to SEQ ID NO: 2 of WO 2016/001449.

Another protease of interest is the alkaline protease from Bacillus lentus DSM 5483, as described for example in WO 91/02792, and variants thereof which are described for example in WO 92/21760, WO 95/23221, EP 1921147, EP 1921148 and WO 2016/096711.

The protease may alternatively be a variant of the TY145 protease having SEQ ID NO: 1 of WO 2004/067737, for example a variant comprising a substitution at one or more positions corresponding to positions 27, 109, 111, 171, 173, 174, 175, 180, 182, 184, 198, 199 and 297 of SEQ ID NO: 1 of WO 2004/067737, wherein said protease variant has a sequence identity of at least 75% but less than 100% to SEQ ID NO: 1 of WO 2004/067737. TY145 variants of interest are described in e.g. WO 2015/014790, WO 2015/014803, WO 2015/014804, WO 2016/097350, WO 2016/097352, WO 2016/097357 and WO 2016/097354.

Examples of Preferred Proteases Include:

(a) variants of SEQ ID NO: 1 of WO 2016/001449 comprising two or more substitutions selected from the group consisting of S9E, N43R, N76D, Q206L, Y209W, S259D and L262E, for example a variant with the substitutions S9E, N43R, N76D, V205I, Q206L, Y209W, S259D, N261W and L262E, or with the substitutions S9E, N43R, N76D, N185E, S188E, Q191N, A194P, Q206L, Y209W, S259D and L262E, wherein position numbers are based on the numbering of SEQ ID NO: 2 of WO 2016/001449;

(b) a variant of the polypeptide of SEQ ID NO: 1 of WO 2016/001449 with the mutation S99SE, wherein position numbers are based on the numbering of SEQ ID NO: 2 of WO 2016/001449;

(c) a variant of the polypeptide of SEQ ID NO: 1 of WO 2016/001449 with the mutation S99AD, wherein position numbers are based on the numbering of SEQ ID NO: 2 of WO 2016/001449;

(d) a variant of the polypeptide of SEQ ID NO: 1 of WO 2016/001449 with the substitutions Y167A+R170S+A194P, wherein position numbers are based on the numbering of SEQ ID NO: 2 of WO 2016/001449;

(e) a variant of the polypeptide of SEQ ID NO: 1 of WO 2016/001449 with the substitutions S9R+A15T+V68A+N218D+Q245R, wherein position numbers are based on the numbering of SEQ ID NO: 2 of WO 2016/001449;

(f) a variant of the polypeptide of SEQ ID NO: 1 of WO 2016/001449 with the substitutions S9R+A15T+G61E+V68A+A194P+V205I+Q245R+N261 D, wherein position numbers are based on the numbering of SEQ ID NO: 2 of WO 2016/001449;

(g) a variant of the polypeptide of SEQ ID NO: 1 of WO 2016/001449 with the substitutions S99D+S101R/E+S103A+V104I+G160S; for example a variant of SEQ ID NO: 1 of WO 2016/001449 with the substitutions S3T+V4I+S99D+S101E+S103A+V104I+G160S+V205I, wherein position numbers are based on the numbering of SEQ ID NO: 2 of WO 2016/001449;

(h) a variant of the polypeptide of SEQ ID NO: 2 of WO 2016/001449 with the substitutions S24G+S53G+S78N+S101N+G128A/S+Y217Q, wherein position numbers are based on the numbering of SEQ ID NO: 2 of WO 2016/001449;

(i) the polypeptide disclosed in GENESEQP under accession number BER84782, corresponding to SEQ ID NO: 302 in WO 2017/210295;

(j) a variant of the polypeptide of SEQ ID NO: 1 of WO 2016/001449 with the substitutions S99D+S101E+S103A+V104I+S156D+G160S+L262E, wherein position numbers are based on the numbering of SEQ ID NO: 2 of WO 2016/001449;

(k) a variant of the polypeptide of SEQ ID NO: 1 of WO 2016/001449 with the substitutions S9R+A15T+G61E+V68A+N76D+S99G+N218D+Q245R, wherein position numbers are based on the numbering of SEQ ID NO: 2 of WO 2016/001449;

(l) a variant of the polypeptide of SEQ ID NO: 1 of WO 2016/001449 with the substitutions V68A+S106A, wherein position numbers are based on the numbering of SEQ ID NO: 2 of WO 2016/001449; and

(m) a variant of the polypeptide of SEQ ID NO: 1 of WO 2004/067737 with the substitutions S27K+N109K+S111E+S171E+S173P+G174K+S175P+F180Y+G182A+L184F+Q198E+N199+T297P, wherein position numbers are based on the numbering of SEQ ID NO: 1 of WO 2004/067737.

Suitable commercially available protease enzymes include those sold under the trade names Alcalase®, Duralase™, Durazym™, Relase®, Relase® Ultra, Savinase®, Savinase® Ultra, Primase™, Polarzyme®, Kannase®, Liquanase®, Liquanase® Ultra, Ovozyme®, Coronase®, Coronase® Ultra, Blaze®, Blaze Evity® 100T, Blaze Evity® 125T, Blaze Evity® 150T, Blaze Evity®200T, Neutrase®, Everlase®, Esperase®, Progress® Uno, Progress® In and Progress® Excel (Novozymes A/S), those sold under the tradename Maxatase™, Maxacal™ Maxapem®, Purafect® Ox, Purafect® OxP, Puramax®, FN2™, FN3™, FN4^{ex}™, Excellase®, Excellenz™ P1000, Excellenz™ P1250, Eraser™, Preferenz® P100, Purafect Prime, Preferenz® P110, Preferenz® P300, Effectenz P1000™, Purafect®, Effectenz P1050™, Purafect® Ox, Effectenz™ P2000, Purafast™, Properase®, Opticlean™ and Optimase® (Danisco/DuPont), BLAP (sequence shown in FIG. 29 of U.S. Pat. No. 5,352,604) and variants hereof (Henkel AG), and KAP (Bacillus alkalophilus subtilisin) from Kao.

### Lipases and Cutinases

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include Lipolase™, Lipex™; Lipolex™ and Lipoclean™ (Novozymes A/S), Lumafast (originally from Genencor) and Lipomax (originally from Gist-Brocades). Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

### Amylases

Suitable amylases may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T49I+G107A+H156Y+A181T+N190F+I201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, I206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T131I+T165I+K178L+T182G+Y305R+G475K wherein the variants are C-terminally truncated and optionally further comprises a substitution at position 243 and/or a deletion at position 180 and/or position 181.

Further suitable amylases are amylases having SEQ ID NO: 1 of WO13184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, I203, S241, R458, T459, D460, G476 and G477. More preferred variants of SEQ ID NO: 1 are those having the substitution in one of more of the following positions: K176L, E187P, N192FYH, M199L, I203YF, S241QADN, R458N, T459S, D460T, G476K and G477K and/or deletion in position R178 and/or S179 or of T180 and/or G181. Most preferred amylase variants of SEQ ID NO: 1 are those having the substitutions:

E187P+I203Y+G476K

E187P+I203Y+R458N+T459S+D460T+G476K

wherein the variants optionally further comprise a substitution at position 241 and/or a deletion at position 178 and/or position 179.

Further suitable amylases are amylases having SEQ ID NO: 1 of WO10104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, I181, G182, M200, L204, E242, G477 and G478. More preferred variants of SEQ ID NO: 1 are those having the substitution in one of more of the following positions: N21D, D97N, V128I K177L, M200L, L204YF, E242QA, G477K and G478K and/or deletion in position R179 and/or S180 or of I181 and/or G182. Most preferred amylase variants of SEQ ID NO: 1 are those having the substitutions:

N21D+D97N+V128I,

wherein the variants optionally further comprise a substitution at position 200 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™ Stainzyme Plus™, Natalase™, Liquozyme X and BAN™ (from Novozymes A/S), and Rapidase™ Purastar™/Effectenz™, Powerase, Preferenz™ S1000, Preferenz™ S100, Preferenz™ 5110 and Preferenz™ S210 (from Genencor International Inc./DuPont).

### Peroxidases/Oxidases

A peroxidase may be an enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity. Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257. A peroxidase may also include a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions. The haloperoxidase may be a chloroperoxidase. Preferably, the haloperoxidase is a vanadium haloperoxidase, i.e., a vanadate-containing haloperoxidase. In a preferred method the vanadate-containing haloperoxidase is combined with a source of chloride ion. Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis. Haloperoxidases have also been isolated from bacteria such as Pseudomonas, e.g., P. pyrrocinia and Streptomyces, e.g., S. aureofaciens. The haloperoxidase may be derivable from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 01/79459, Dendryphiella salina as described in WO 01/79458, Phaeotrichoconis crotalarie as described in WO 01/79461, or Geniculosporium sp. as described in WO 01/79460.

Oxidases include any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5). Preferred laccase enzymes are enzymes of microbial origin. The enzymes may be derived from plants, bacteria or fungi (including filamentous fungi and yeasts). Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Scytalidium, e.g., S. thermophilum, Polyporus, e.g., P. pinsitus, Phlebia, e.g., P. radiata (WO 92/01046), or Coriolus, e.g., C. hirsutus (JP 2238885). Suitable examples from bacteria include a laccase derivable from a strain of Bacillus. A laccase derived from Coprinopsis or Myceliophthora is preferred; in particular a laccase derived from Coprinopsis cinerea, as disclosed in WO 97/08325; or from Myceliophthora thermophila, as disclosed in WO 95/33836.

### Microorganisms

The detergent additive as well as the detergent composition may also comprise one or more microorganisms, such as one or more fungi, yeast, or bacteria. In an embodiment, the one or more microorganisms are dehydrated (for example by lyophilization) bacteria or yeast, such as a strain of Lactobacillus. In another embodiment, the microorganisms are one or more microbial spores (as opposed to vegetative cells), such as bacterial spores; or fungal spores, conidia, hypha. Preferably, the one or more spores are Bacillus endospores; even more preferably the one or more spores are endospores of Bacillus subtilis, Bacillus licheniformis, Bacillus amyloliquefaciens, or Bacillus megaterium. The microorganisms may be included in the detergent composition or additive in the same way as enzymes (see above).

### Formulation of Detergent Products

The cleaning composition of the present invention may be formulated, for example, as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations. In a specific aspect, the present invention provides a detergent additive comprising one or more enzymes as described herein. The cleaning composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multicompartments. It can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water-soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water-soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water-soluble film. The compartment for liquid components can be different in composition than compartments containing solids: US2009/0011970 A1.

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent which is not unit dosed may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent. A liquid or gel detergent may also be non-aqueous.

### Formulation of Enzyme in Granules

Non-dusting granulates may be produced e.g. as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono-, di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

The compositions of the invention may be formulated as a granule, for example as a co-granule that combines one or more enzymes. Each enzyme will then be present in more granules, securing a more uniform distribution of enzymes in the detergent. This also reduces the physical segregation of different enzymes due to different particle sizes. Methods for producing multi-enzyme co-granulates for the detergent industry are disclosed in the IP.com disclosure IPCOM000200739D.

Another example of formulation of enzymes by the use of co-granulates is disclosed in WO 2013/188331, which relates to a detergent composition comprising (a) a multi-enzyme co-granule; (b) less than 10 wt % zeolite (anhydrous basis); and (c) less than 10 wt % phosphate salt (anhydrous basis), wherein said enzyme co-granule comprises from 10 to 98 wt % moisture sink component and the composition additionally comprises from 20 to 80 wt % detergent moisture sink component. WO 2013/188331 also relates to a method of treating and/or cleaning a surface, preferably a fabric surface comprising the steps of (i) contacting said surface with the detergent composition in aqueous wash liquor, (ii) rinsing and/or drying the surface.

A multi-enzyme co-granule may comprise an enzyme of the invention and one or more enzymes selected from the group consisting of proteases, lipases, cellulases, xyloglucanases, perhydrolases, peroxidases, lipoxygenases, laccases, hemicellulases, proteases, cellulases, cellobiose dehydrogenases, xylanases, phospho lipases, esterases, cutinases, pectinases, mannanases, pectate lyases, keratinases, reductases, oxidases, phenoloxidases, ligninases, pullulanases, tannases, pentosanases, lichenases glucanases, arabinosidases, hyaluronidase, chondroitinase, amylases, nucleases, hexosaminidases and mixtures thereof.

An embodiment of the invention relates to an enzyme granule/particle comprising the enzyme of the invention. The granule is composed of a core, and optionally one or more coatings (outer layers) surrounding the core. Typically, the granule/particle size, measured as equivalent spherical diameter (volume based average particle size), of the granule is 20-2000 μm, particularly 50-1500 μm, 100-1500 μm or 250-1200 μm.

The core may include additional materials such as fillers, fibre materials (cellulose or synthetic fibres), stabilizing agents, solubilising agents, suspension agents, viscosity regulating agents, light spheres, plasticizers, salts, lubricants and fragrances.

The core may include binders, such as synthetic polymer, wax, fat, or carbohydrate.

The core may comprise a salt of a multivalent cation, a reducing agent, an antioxidant, a peroxide decomposing catalyst and/or an acidic buffer component, typically as a homogenous blend.

The core may consist of an inert particle with the enzyme absorbed into it, or applied onto the surface, e.g., by fluid bed coating.

The core may have a diameter of 20-2000 μm, particularly 50-1500 μm, 100-1500 μm or 250-1200 μm.

The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation.

Methods for preparing the core can be found in Handbook of Powder Technology; Particle size enlargement by C. E. Capes; Volume 1; 1980; Elsevier.

The core of the enzyme granule/particle may be surrounded by at least one coating, e.g., to improve the storage stability, to reduce dust formation during handling, or for coloring the granule. The optional coating(s) may include a salt coating, or other suitable coating materials, such as polyethylene glycol (PEG), methyl hydroxy-propyl cellulose (MHPC) and polyvinyl alcohol (PVA). Examples of enzyme granules with multiple coatings are shown in WO 93/07263 and WO 97/23606.

The coating may be applied in an amount of at least 0.1% by weight of the core, e.g., at least 0.5%, 1% or 5%. The amount may be at most 100%, 70%, 50%, 40% or 30%.

The coating is preferably at least 0.1 μm thick, particularly at least 0.5 μm, at least 1 μm or at least 5 μm. In a particular embodiment, the thickness of the coating is below 100 μm. In a more particular embodiment the thickness of the coating is below 60 μm. In an even more particular embodiment the total thickness of the coating is below 40 μm.

The coating should encapsulate the core unit by forming a substantially continuous layer. A substantially continuous layer is to be understood as a coating having few or no holes, so that the core unit it is encapsulating/enclosing has few or no uncoated areas. The layer or coating should be homogeneous in thickness.

The coating can further contain other materials as known in the art, e.g., fillers, antisticking agents, pigments, dyes, plasticizers and/or binders, such as titanium dioxide, kaolin, calcium carbonate or talc.

A salt coating may comprise at least 60% by weight w/w of a salt, e.g., at least 65%, at least 70%, at least 75%, at least 80%, at least 85%, at least 90%, at least 95% or at least 99% by weight w/w.

The salt may be added from a salt solution where the salt is completely dissolved or from a salt suspension wherein the fine particles is less than 50 μm, such as less than 10 μm or less than 5 μm.

The salt coating may comprise a single salt or a mixture of two or more salts. The salt may be water soluble, in particular having a solubility at least 0.1 grams in 100 g of water at 20° C., preferably at least 0.5 g per 100 g water, e.g., at least 1 g per 100 g water, e.g., at least 5 g per 100 g water.

The salt may be an inorganic salt, e.g., salts of sulfate, sulfite, phosphate, phosphonate, nitrate, chloride or carbonate or salts of simple organic acids (less than 10 carbon atoms, e.g., 6 or less carbon atoms) such as citrate, malonate or acetate. Examples of cations in these salts are alkali or earth alkali metal ions, the ammonium ion or metal ions of the first transition series, such as sodium, potassium, magnesium, calcium, zinc or aluminium. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, lactate, formate, acetate, butyrate, propionate, benzoate, tartrate, ascorbate or gluconate. In particular alkali- or earth alkali metal salts of sulfate, sulfite, phosphate, phosphonate, nitrate, chloride or carbonate or salts of simple organic acids such as citrate, malonate or acetate may be used.

The salt in the coating may have a constant humidity at 20° C. above 60%, particularly above 70%, above 80% or above 85%, or it may be another hydrate form of such a salt (e.g., anhydrate). The salt coating may be as described in WO 00/01793 or WO 2006/034710.

Specific examples of suitable salts are NaCl (CH_{20° C.}=76%), Na₂CO₃ (CH_{20° C.}=92%), NaNO₃ (CH_{20° C.}=73%), Na₂HPO₄ (CH_{20° C.}=95%), Na₃PO₄ (CH_{25° C.}=92%), NH₄Cl (CH_{20° C.}=79.5%), (NH₄)₂HPO₄ (CH_{20° C.}=93.0%), NH₄H₂PO₄ (CH_{20° C.}=93.1%), (NH₄)₂SO₄ (CH_{20° C.}=81.1%), KCl (CH_{20° C.}=85%), K₂HPO₄ (CH_{20° C.}=92%), KH₂PO₄ (CH_{20° C.}=96.5%), KNO₃ (CH_{20° C.}=93.5%), Na₂SO₄ (CH_{20° C.}=93%), K₂SO₄ (CH_{20° C.}=98%), KHSO₄ (CH_{20° C.}=86%), MgSO₄ (CH_{20° C.}=90%), ZnSO₄ (CH_{20° C.}=90%) and sodium citrate (CH_{25° C.}=86%). Other examples include NaH₂PO₄, (NH₄)H₂PO₄, CuSO₄, Mg(NO₃)₂ and magnesium acetate.

The salt may be in anhydrous form, or it may be a hydrated salt, i.e. a crystalline salt hydrate with bound water of crystallization, such as described in WO 99/32595. Specific examples include anhydrous sodium sulfate (Na₂SO₄), anhydrous magnesium sulfate (MgSO₄), magnesium sulfate heptahydrate (MgSO₄.7H₂O), zinc sulfate heptahydrate (ZnSO₄.7H₂O), sodium phosphate dibasic heptahydrate (Na₂HPO₄.7H₂O), magnesium nitrate hexahydrate (Mg(NO₃)₂(6H₂O)), sodium citrate dihydrate and magnesium acetate tetrahydrate. Preferably the salt is applied as a solution of the salt, e.g., using a fluid bed.

Thus, in a further aspect, the present invention provides a granule comprising:

(a) a core comprising an enzyme according to the invention,

(b) optionally, a coating consisting of one or more layers surrounding the core; and

(c) wherein the granule preferably is a co-granulate comprising one or more additional enzymes, preferably wherein at least one additional enzyme is selected from proteases, amylases and cellulases.

In one embodiment, the present invention provides a granule comprising:

(a) a core comprising a polypeptide having fructan degrading activity,

(b) optionally, a coating consisting of one or more layers surrounding the core; and

(c) wherein the granule preferably is a co-granulate comprising one or more additional enzymes, preferably wherein at least one additional enzyme is selected from proteases, amylases and cellulases.

In one embodiment, the present invention provides a granule comprising:

(a) a core comprising a polypeptide having fructan degrading activity, wherein the polypeptide is selected from the group consisting of: SEQ ID NO: 3, SEQ ID NO: 6, SEQ ID NO: 9, SEQ ID NO: 12, SEQ ID NO: 15 and SEQ ID NO: 18, and polypeptides having at least 60% sequence identity, e.g., at least 65%, at least 70%, at least 75%, at least 80%, at least 85%, at least 90%, at least 91%, at least 92%, at least 93%, at least 94%, at least 95%, at least 96%, at least 97%, at least 98% or at least 99% sequence identity hereto,

(b) optionally, a coating consisting of one or more layers surrounding the core; and

(c) wherein the granule preferably is a co-granulate comprising one or more additional enzymes, preferably wherein at least one additional enzyme is selected from proteases, amylases and cellulases.

### Uses

The present invention is also directed to methods for using the compositions thereof for cleaning of laundry/textiles/fabrics, including household laundry and industrial laundry, and for hard surface cleaning, including automatic dishwashing (ADW), car washing and industrial surface cleaning.

### Use of Cleaning Compositions

The detergent composition of the present invention may be formulated, for example, for use as a hand or machine laundry detergent composition, including as a laundry additive composition suitable for pre-treatment of stained fabrics or a rinse added to a fabric softener composition, or be formulated as a detergent composition for use in general household hard surface cleaning operations, or be formulated for hand or machine dishwashing. Also provided herein is a detergent additive comprising one or more fructan degrading enzymes described herein and optionally a nuclease such as a DNase.

In one embodiment, the invention relates to use of a cleaning composition comprising a fructan degrading enzyme as described herein and optionally a nuclease such as a DNase for cleaning of an item, wherein the item is a textile or a surface.

In another embodiment, the invention relates to use of a fructan degrading enzyme as described herein, optionally in combination with a nuclease such as a DNase, for removing biofilm from an item, such as a textile or a surface.

### Methods

The invention further relates to a method of treating a fabric, for example for removing biofilm from the fabric, the method comprising;
a) contacting the fabric with an aqueous solution of a fructan degrading enzyme, and optionally with at least one nuclease polypeptide having DNase or RNase activity; and optionally
b) rinsing and drying the fabric.

The invention further relates to a method for cleaning or laundering an item comprising the steps of:
a) exposing an item to a wash liquor comprising at least one fructan degrading enzyme, or a detergent composition comprising such enzyme, and optionally to at least one nuclease polypeptide having DNase or RNase activity;
b) completing at least one wash cycle; and optionally
c) rinsing the item,
   wherein the item is a fabric.

The invention further relates to a method for cleaning or laundering an item comprising the steps of:
a) exposing an item to a wash liquor comprising a polypeptide or a detergent composition comprising a polypeptide, wherein the polypeptide is selected from the group consisting of: SEQ ID NO: 3, SEQ ID NO: 6, SEQ ID NO: 9, SEQ ID NO: 12, SEQ ID NO: 15 and SEQ ID NO: 18, and polypeptides having at least 60% sequence identity, e.g., at least 65%, at least 70%, at least 75%, at least 80%, at least 85%, at least 90%, at least 91%, at least 92%, at least 93%, at least 94%, at least 95%, at least 96%, at least 97%, at least 98% or at least 99% sequence identity hereto, and optionally to at least one nuclease polypeptide having DNase or RNase activity;
b) completing at least one wash cycle; and optionally
c) rinsing the item, wherein the item is a fabric.

The pH of the liquid solution is typically in the range of from about 5.5 to about 12, such as from about 7 to about 11, such as from about 7 to about 10, e.g. from about 7 to about 9.

The wash liquor may have a temperature in the range of 5° C. to 95° C., such as in the range of 10° C. to 80° C., in the range of 10° C. to 70° C., in the range of 10° C. to 60° C., in the range of 10° C. to 50° C., in the range of 15° C. to 40° C. or in the range of 20° C. to 30° C. In one aspect, the temperature of the wash liquor is about 30° C.

The concentration of the fructan degrading enzyme in the wash liquor is typically in the range of at least 0.00001 ppm to at least 10 ppm, at least 0.00002 ppm to at least 10 ppm, at least 0.0001 ppm to at least 10 ppm, at least 0.0002 ppm to at least 10 ppm, at least 0.001 ppm to at least 10 ppm, at least 0.002 ppm to at least 10 ppm, at least 0.01 ppm to at least 10 ppm, at least 0.02 ppm to at least 10 ppm, at least 0.1 ppm to at least 10 ppm, at least 0.2 ppm to at least 10 ppm, at least 0.5 ppm to at least 5 ppm.

The present invention is further described by the following examples that should not be construed as limiting the scope of the invention.

### EXAMPLES

### Example 1: Wash Assay

### Preparation of Biofilm Swatches

Biofilm swatches (10 cm×10 cm) were made by growing Brevundimonas sp. on polyester swatches for three days. The biofilm swatches were rinsed twice in water and dried for 2 h in a lab bench with flow and subsequently punched into round swatches (0.6 cm diam.) and placed into the wells of an MTP96 microtiter plate, and stored at 4° C. for further use.

### Wash Experiment

Wash performance of fructanase enzymes of the invention was screened alone and together with a DNase. The biofilm swatches were transferred from the MTP96 plate to a deep-well MTP96 plate for the wash assay. The deep-well plate was placed in a Hamilton robot and subjected to a wash simulation program using the following conditions: shaking speed: 30 sec at 1000 rpm; duration of wash cycle: 30 minutes with shaking; temperature 30° C.; total volume of wash liquor: 500 μl per well (490 μl wash liquor of Model detergent A+10 μl sample).

The Model detergent A wash liquor was prepared by dissolving 3.3 g/L of the detergent in water with a hardness of 15° dH. Soil (WFK 09V pigment soil; Testgewebe GmbH, Germany) was subsequently added to reach a concentration of 0.7 g soil/L.

TABLE 1
Composition of Model detergent A

| | Content of active |
|---|---|
| Ingredient | component (% w/w) |
| LAS (linear alkylbenzene sulfonate) | 12 |
| AEO (alcohol ethoxylate) | 11 |
| AEOS (alcohol ether sulfate), SLES (sodium | 5 |
| lauryl ether sulfate) | |
| MPG (propane-1,2-diol) | 6 |
| Ethanol | 3 |
| TEA (2,2′,2″-nitrilotriethanol) | 3 |
| Coco fatty acid | 2.75 |
| Soy fatty acid | 2.75 |
| Glycerol | 2 |
| Sodium hydroxide (solid crystals) | 2 |
| Sodium citrate | 2 |
| Sodium formate | 1 |
| DTMPA (diethylenetriamine | 0.2 |
| penta(methylene phosphonic acid) | |
| PCA (copoly(acrylic acid/maleic acid), | 0.2 |
| sodium salt) | |
| Water (ion-exchanged) | to 100% |

A 96 deep-well plate was filled with each enzyme sample, and the program was started on the robot. The fructanases were tested in a concentration 0.5 ppm, with and without a low dose (0.00001 ppm) of a DNase from Bacillus cibi (SEQ ID NO: 19). The enzyme samples were tested against a blank consisting of biofilm swatches without any enzyme. After completion of the wash simulation cycle, the swatches were removed from the wash liquor and dried on filter paper. The dried swatches were fixed on a sheet of white paper for scanning, and the scanned pictures were analyzed with color-analyzer software. Each sample has an intensity measurement from the color analyzer software analysis that is used to calculate the delta intensity (remission) compared to the blank, by subtracting the intensity value of the blank without enzyme from the intensity value of a sample with enzyme. Values over 20 are visible to the human eye.

The remission value of the blank in this experiment was 168.

Table 2 below shows the wash performance of seven fructan degrading enzymes of the invention, with the measured remission intensity for the single enzymes and delta intensity compared to blank, as well as the measured intensity for the combination of two enzymes (0.5 ppm fructanase+0.00001 ppm DNase) and delta intensity for the combinations of two enzymes compared to blank. The intensity and delta intensity of a sample with the DNase alone is also given.

It may be seen that all of the fructanase enzymes are able to provide improved removal of biofilm from the swatches, and furthermore that a clear synergistic effect is obtained using a combination of a fructanase and a low dose of DNase.

TABLE 2
Wash performance on biofilm of fructanases with and without DNase

| | | Delta | | Delta |
|---|---|---|---|---|
| | Intensity | intensity | Intensity | intensity |
| | (one | (one | (two | (two |
| Enzyme | enzyme) | enzyme) | enzymes) | enzymes) |
| SEQ ID NO: 3 | 176 | 8 | 234 | 66 |
| SEQ ID NO: 6 | 193 | 25 | 246 | 78 |
| SEQ ID NO: 9 | 182 | 14 | 253 | 85 |
| SEQ ID NO: 12 | 197 | 29 | 246 | 78 |
| SEQ ID NO: 15 | 193 | 25 | 228 | 60 |
| SEQ ID NO: 18 | 190 | 22 | 247 | 79 |
| SEQ ID NO: 19 (DNase) | 181 | 13 | — | — |

### Example 2: Fructanase Activity Assay

The fructanase enzymes were incubated with the insoluble fructan substrate K-FRUCHK (Megazyme) as follows: 3 mg of fructan (Megazyme K-FRUCHK) was weighed into one Eppendorf tube per sample. Each sample was diluted 10× in 100 mM Na-Acetate, pH 4.5 (with the exception of the SEQ ID NO: 12 sample, which was already diluted and therefore was pipetted directly). Samples were added to a final concentration of 100 ppm (0.1 mg/mL)

| Sample | μL sample | μL 25 mM Na-Acetate, |
|---|---|---|
| SEQ ID NO: | (diluted 10x) | pH 4.5 |
| 6 | 51 | 249 |
| 9 | 68 | 232 |
| 12 | 32 | 268 |
| 15 | 85 | 215 |
| 18 | 34 | 266 |
| Blank (Fructan alone) | — | 300 |

Samples containing enzyme plus substrate were incubated for 60 min at 37° C. at 1400 rpm, and one sample without enzyme addition was used as a control. After incubation, samples were spinned down for 5 min at 16100×G at room temperature. The supernatants were analyzed by 1) liquid chromatography and 2) reducing ends assay, as follows:

Liquid chromatography was performed on a Dionex™-IC300 system with a PD-10 column (Thermo Fisher Scientific) using the following gradient:

| time | flow | % | % NAOH | % NaAcO |
|---|---|---|---|---|
| (min) | (mL/min) | Water | 0.5M | 0.5M |
| 0 | 0.8 | 97 | 3 | 0 |
| 4.5 | 0.8 | 96 | 4 | 0 |
| 7 | 0.8 | 93 | 7 | 0 |
| 10 | 0.8 | 80 | 15 | 5 |
| 25 | 0.8 | 50 | 15 | 35 |
| 28.1 | 0.8 | 85 | 15 | 0 |
| 29 | 0.8 | 90 | 10 | 0 |
| 30.1 | 0.8 | 97 | 3 | 0 |
| 33 | 0.8 | 97 | 3 | 0 |
| 33.1 | 0 | — | — | — |

Fructose and glucose (Sigma) were used as standard references.

For the reducing sugars assay, the working buffer was prepared by weighing 50 g of potassium sodium tartrate (K—Na-tartrate, Merck 8087) and 20 g of NaOH (Merck 1.06498) into 1 L of water.

For the reducing agent, also called PAHBAH reagent, a solution of PAHBAH (4-Hydroxybenzoic hydrazide, Sigma H-9882) was prepared by weighing 225 mg PAHBAH into 15 ml of buffer.

The colorimetric reaction was made by transferring 75 μL of the respective sample supernatant to a PCR plate and by adding 150 μL PAHBAH reagent.

After incubation at 95° C. for 10 minutes in the PCR machine, 150 μl of each sample was transferred to a microtiter plate to read the absorbance at 405 nm.

### Results

Liquid chromatography showed that the fructanases are active on fructan. However, the two enzymes with SEQ ID NO: 12 and SEQ ID NO: 18 gave different hydrolysis products than the others. These enzymes gave monosaccharides, fructose and glucose, suggesting exo-activity. The other enzymes tested here gave oligosaccharide profiles of different sizes, suggesting endo-activity.

The table below describes monosaccharides released by liquid chromatography measured as Area in nano coulombs per minute (nC*min). The two sugars identified are glucose and fructose. For the other fructo-oligosaccharides the degree of polymerization (dp) is in the range of 1-10.

| | Area (nC*min) Glucose + Fructose | | | | |
|---|---|---|---|---|---|
| Time | SEQ ID | SEQ ID | SEQ ID | SEQ ID | SEQ ID |
| (min) | NO: 6 | NO: 9 | NO: 12 | NO: 15 | NO: 18 |
| 10.7 | 0 | 0 | 85.8 | 0 | 78.9 |
| 12.1 | 50.2 | 86.9 | 540.5 | 142.2 | 347.5 |
| 12.4 | 0 | 0 | 40.1 | 0 | 151.2 |
| 13 | 27.6 | 46.2 | 38.1 | 41.7 | 0 |
| 13.6 | 11.4 | 13.2 | 71.3 | 10.5 | 64.7 |
| 17.4 | 45.7 | 61.2 | 22.1 | 78.3 | 84.3 |
| 17.9 | 28.6 | 27.2 | 0 | 23.7 | 0 |
| 18.5 | 131 | 153.8 | 0 | 152.5 | 15.8 |
| 19.7 | 0 | 0 | 0 | 0.3 | 0 |
| 20 | 18.2 | 0.2 | 0 | 0 | 0 |
| 20.1 | 77 | 49.8 | 0 | 0 | 0 |
| 21.6 | 27.3 | 18.2 | 0 | 0 | 0 |

The level of reducing sugars with SEQ ID NO: 12 and SEQ ID NO: 18 was also consistent with the results in the table above, giving a higher level of activity for these two enzymes.

The absorbance at 405 nm reflects activity on fructan and was as follows:

| SEQ ID NO: | Abs 405 nm |
|---|---|
| 6 | 1.83 |
| 9 | 1.94 |
| 12 | 2.70 |
| 15 | 2.02 |
| 18 | 2.55 |
| no enzyme | 0.21 |

### Example 3: Construction of Clades and Phylogenetic Trees

GH32 Phylogenetic Tree

A phylogenetic tree was constructed from polypeptide sequences of the invention containing a GH32 domain, as defined in CAZY (Lombard, Henrissat et al, 2014. The carbohydrate-active enzymes database (CAZy), in 2013, Nucleic Acids Res. 42, http://www.cazy.org/). The phylogenetic tree was constructed from a multiple alignment of mature polypeptide sequences containing at least one GH32 domain. The sequences were aligned using the MUSCLE algorithm version 3.8.31 (Edgar, 2004. Nucleic Acids Research 32(5): 1792-1797), and trees were constructed using FastTree version 2.1.8 (Price et al., 2010, PloS one 5(3)) and visualized using iTOL (Letunic & Bork, 2007. Bioinformatics 23(1): 127-128).

A subset of polypeptides containing a GH32 domain also contains a glycosyl hydrolase family 32 C terminal domain (GH32C), as defined by Pfam domain ID PF08244 (The Pfam protein families database: towards a more sustainable future, Finn et al., Nucleic Acids Research (2016) Database Issue 44:D279-D285). The polypeptides of the invention contain a GH32 domain, as well as a glycosyl hydrolase family 32 C terminal domain. The glycosyl hydrolase family 32 C terminal domain will be denoted the GH32C domain. As an example, in SEQ ID NO: 6 from Aspergillus deflectus, the GH32C domain is located at positions 351 to 484.

Generation of Clades

In addition to containing a GH32 domain, as well as a GH32C domain, the polypeptides of the invention also comprise one or more of several motifs.

One example is the motif [WPG][GMTH][NH][AEILDNMV] (SEQ ID NO: 24) situated in positions corresponding to positions 15 to 18 in Aspergillus deflectus (SEQ ID NO: 6). Preferably, the sequence of the motif in these positions is W[GMT]N[NDEI] (SEQ ID NO: 32).

The polypeptides containing a GH32 domain can be separated into distinct sub-clusters, or clades, which are defined by one or more short sequence motifs, as well as containing a GH32 domain and a GH32C domain.

We denoted the sub-cluster comprising the motif [WPG][GMTH][NH][AEILDNMV] (SEQ ID NO: 24) as the WMNE clade. Preferably, this motif has the sequence W[GMT]N[NDEI] (SEQ ID NO: 32). Polypeptide sequences containing a GH32 domain, a GH32C domain, as well as the motif of SEQ ID NO: 24, preferably the motif of SEQ ID NO: 32, will be denoted as belonging to the WMNE clade.

The polypeptides in the WMNE clade can be further separated into multiple distinct sub-clusters, or clades. The distinct motifs for each of these clades are described in detail below.

Generation of the WMNE Clade

A phylogenetic tree was constructed from polypeptide sequences containing a GH32 domain as defined above from a multiple alignment of mature polypeptide sequences containing at least one GH32 domain, using the MUSCLE algorithm and FastTree, and visualizing using iTOL. Using the phylogenetic tree, the polypeptides in GH32 can be separated into distinct sub-clusters, one which we denoted WMNE.

A characteristic motif for this sub-cluster is the motif [WPG][GMTH][NH][AEILDNMV] (SEQ ID NO: 24), corresponding to amino acids WMNE at positions 15 to 18 in Aspergillus deflectus (SEQ ID NO: 6). Preferably, the sequence of the motif in these positions is W[GMT]N[NDEI] (SEQ ID NO: 32).

Polypeptides containing a GH32 domain, a GH32C domain, as well as the motif, belong to the WMNE clade.

The motif of SEQ ID NO: 24 is preferably W[GMT]N[NDEI] (SEQ ID NO: 32). in some embodiments more particularly WMN[DE] (SEQ ID NO: 30), for example WMN[DE]PNG (SEQ ID NO: 31). Examples of polypeptides of the WMNE clade having these specific motifs are SEQ ID NO: 6, 9, 12, 15 and 18.

The WMNE can be split into sub-clusters, one denoted the AYSN clade and one denoted the WMNEP clade.

Generation of the AYSN Clade

The polypeptides of the AYSN clade contain a GH32 domain, belong to the WMNE clade, and comprise the motif A[YF]S[LN]D[QK] (SEQ ID NO: 25), corresponding to amino acids AYSNDK (SEQ ID NO: 26) at positions 120 to 125 of Bacillus licheniformis (SEQ ID NO: 18), where D corresponding to position 124 is fully conserved in the AYSN clade.

Another motif contained in the polypeptides of the AYSN clade is [HS]WGH (SEQ ID NO: 27), located at positions 53 to 56 in Bacillus licheniformis (SEQ ID NO: 18).

Examples of polypeptides of the AYSN clade include SEQ ID NO: 12 and SEQ ID NO: 18.

Generation of the WMNEP Clade

The WMNE clade can be split into another, separate sub-cluster, denoted the WMNEP clade. Polypeptides of the WMNEP clade comprise the motif [QLKEYMRVTANI][NSYHATRD][WF][MEVKTL] [NGA][EVDLIH][PAEHDS][NATSKGVERQCD] (SEQ ID NO: 28), corresponding to amino acids KNWMNEPN (SEQ ID NO: 29) at positions 13 to 20 of Aspergillus deflectus (SEQ ID NO: 6).

Examples of polypeptides of the WMNEP clade include SEQ ID NO: 6, SEQ ID NO: 9, and SEQ ID NO: 15.

1. A cleaning composition comprising a GH32 fructan degrading enzyme and at least one detergent component, wherein the fructan degrading enzyme comprises a GH32 domain and optionally a GH32C domain.

2. (canceled)

3. The cleaning composition of claim 1, wherein the fructan degrading enzyme belongs to the WMNE clade and comprises the motif W[GMT]N[NDEI] (SEQ ID NO: 32).

4. The cleaning composition of claim 1, wherein the fructan degrading enzyme belongs to the AYSN clade and comprises the motif A[YF]S[LN]D[QK] (SEQ ID NO: 25) and/or the motif [HS]WGH (SEQ ID NO: 27); and/or wherein the fructan degrading enzyme belongs to the WMNEP clade and comprises the motif [QLKEYMRVTANI] [NSYHATRD][WF][MEVKTL][NGA][EVDLIH][PAEHDS][NATSKGVERQCD](SEQ ID NO: 28).

5. The cleaning composition of claim 1, wherein the fructan degrading enzyme is selected from the group consisting of:
a) a polypeptide having a sequence identity to SEQ ID NO: 6 of at least 60%;
b) a polypeptide having a sequence identity to SEQ ID NO: 9 of at least 60%;
c) a polypeptide having a sequence identity to SEQ ID NO: 12 of at least 60%;
d) a polypeptide having a sequence identity to SEQ ID NO: 15 of at least 60%;
e) a polypeptide having a sequence identity to SEQ ID NO: 18 of at least 60%; and
f) a fragment of the polypeptide of (a), (b), (c), (d), (e), (f) or (g), wherein said fragment has fructan degrading activity.

6. The cleaning composition of claim 1, wherein the fructan degrading enzyme has one or more enzyme activities selected from the group consisting of fructan β(2,1)-fructosidase activity (EC 3.2.1.153), fructan β(2,6)-fructosidase activity (EC 3.2.1.154), fructan β-fructosidase activity (EC 3.2.1.80), levanase activity (EC 3.2.1.65), 2,6-β-fructan 6-levanbiohydrolase activity (EC 3.2.1.64) and inulinase activity (EC 3.2.1.7).

7. The cleaning composition of claim 1, comprising at least one surfactant, and optionally: (a) at least one additional cleaning component selected from builders and bleach components, and/or (b) at least one additional enzyme.

8. The cleaning composition of claim 1, further comprising at least one nuclease polypeptide having DNase or RNase activity.

9. The cleaning composition of claim 8, wherein the nuclease is a polypeptide having DNase activity selected from the group consisting of:
a) a polypeptide having a sequence identity to SEQ ID NO: 19 of at least 60%;
b) a polypeptide having a sequence identity to SEQ ID NO: 20 of at least 60%;
c) a polypeptide having a sequence identity to SEQ ID NO: 21 of at least 60%;
d) a polypeptide having a sequence identity to SEQ ID NO: 22 of at least 60%;
e) a polypeptide having a sequence identity to SEQ ID NO: 23 of at least 60%; and
f) a fragment of the polypeptide of (a), (b), (c), (d) or (e), wherein said fragment has DNase activity.

10-11. (canceled)

12. A method of cleaning an item, comprising contacting the item with a solution comprising a fructan degrading enzyme and at least one cleaning component, and optionally with at least one nuclease polypeptide having DNase or RNase activity.

13. A polypeptide having fructanase activity, wherein the polypeptide comprises a GH32 domain and optionally a GH32C domain; and wherein the polypeptide belongs to the WMNE clade and comprises the motif W[GMT]N[NDEI] (SEQ ID NO: 32);
and optionally wherein the polypeptide belongs to the AYSN clade and comprises the motif A[YF]S[LN]D[QK] (SEQ ID NO: 25) and/or the motif [HS]WGH (SEQ ID NO: 27), and/or wherein the polypeptide belongs to the WMNEP clade and comprises the motif [QLKEYMRVTANI][NSYHATRD][WF][MEVKTL][NGA][EVDLIH][PAEHDS][NATSKGVERQCD](SEQ ID NO: 28).

14. The polypeptide of claim 13, wherein the polypeptide is selected from the group consisting of:
(a) a polypeptide having at least 60%;
(b) a polypeptide having at least 60%;
(c) a polypeptide having at least 60%;
(d) a polypeptide having at least 60%; and
(e) a polypeptide having at least 60%.

15. The polypeptide of claim 13, wherein the polypeptide has one or more enzyme activities selected from the group consisting of fructan β(2,1)-fructosidase activity (EC 3.2.1.153), fructan β(2,6)-fructosidase activity (EC 3.2.1.154), fructan β-fructosidase activity (EC 3.2.1.80), levanase activity (EC 3.2.1.65), 2,6-β-fructan 6-levanbiohydrolase activity (EC 3.2.1.64) and inulinase activity (EC 3.2.1.7).


SEQUENCE LISTING (32 sequences)

SEQ ID NO: 1 - DNA, 2769 bp, Paenibacillus amylolyticus
  CDS (1)..(2769)
  sig_peptide (1)..(93)
  mat_peptide (94)..(2769)
atgaaagataaatctaaattcgcaatatgtcttctgatggcaacgggattaactctgacttcaaacggtatttttacatctaaatcaattgcggctagtgctgtacttgctgatcaggaaacaaataaacaggctaaggaggagcttgatatttttgaaagcgcatccaagtcaaataccaatctgataggctggcaagtgaaaggaaaaggaggattagaggatacttcagaaggaatcttgctaacttcacagcctaaagaaaatgtaatggccatctcagagacagtgtccgatgattttatctatgaagctgatgtcatgatcagggatacgaaggcagacgcaacattgttgtttcgttctaatgaggatggctttagctcgtacatgctgcaaatcgttccggacgcaggtctgattcggttaagagatgcaagaactggagatgggaaattaaaggaggagcgtaaagtttctgttgaaatggggcaaatctatcatctcaaagtgaaggcagtgggttcctcgctaaaagtatattggggcaatcaatataaaccattaattgatgttcaagacatttcgtatcaaagcggaaagcttggactccatgtatgggatggatccgccttgttttcgaacatcgtggtgagcgatctgaaaggtaatttgggtacggtgctttctagcaagggaaaatggcagcctgatatcaacggcaaaagagggacggtagaacaggggagcatagcrcagcaaatctataacaaagaggcgactgatatggtctatgaagggaatattacccttcgtcctgattctattgcagctctcgcatttcgatcttcaaccgatggagctgaaggatatgaagctactcttacgaaggaaggagatcaggtccgtgtaagtttgacgaatacaaaaggaacagtaattgcaagttcgcaacgtacttatccgagtcagatgggagccaaacatcatgtggaaatcaaggcaaagggagatcgaattcaggtcttcctggacgggtacactacggccgcgattgacgtgaaagattcaacctacaagagtggaagtaccggaatcgtcgtaaaaaaggggacggcttactttcaggatacttatgtgacggaactgagccaatattacaatgagatatatcgtccacaatatcactacacgccaatacgtggttcagcgagtgatccgaatggacttgtctacttcgaaggagaatatcatctcttccatcaggatggaggaacatgggcgcatgcggtaagtaaagatatgctgaactggaaacggcttccgattgcacttccctggaatgatcacggacatgtctggtctgggtcggctgttgcggatatgacaaatgcatctggtttattcggagattcaggaggcaagggccttattgcatactatacttcctttaatccggatagcccgaatgggaaccagcgtataggtctagcttacagtaaagaccaaggtcgtacttgggagtattcgaaggagcgcccgattgtgatcgagaacccgggtaagagcggaaatgaagctgggaattgggatttccgcgatccgaaagtaatccgtgatgatgagaataaccgatgggttatggttgtgtccggaggagatcatattcgtttctatacttcaacaaatttacttgactggacattgacagataattggggatatggggattacgtccgcggaggagtatgggaatgccctgatttgttccagcttccggtagacggaacgtcacagaaaaagtgggttatgatgatcagtacaggagcgaatcccaaaacaggcggatcagatgccgagtattttatcggtcatttaacagctgatggtaaattcgtaaacgataatccggcaggtaaggtgttaagaacagattttggtaaagaattttacgcttcgatgtcttttgctaacatgcctgatcatcgcacagtgatgatggcgtggatgacgaattgggattatccgttcgctttcccaacgtccaattggaaaggtgaactaaccattccgagagaagtatcattggtaacgaccgaagatggaattcggatggtgcaaagtccaatcaaagaattggaatcactgcgtaaacctttgtattctgcttctaacaagtcggtgagcccttcttccgggaatctgctaaaaggtattatttcaggtgcttacgaaattgaagctgaaattgaaatccctgaaaccagcacagtgaccgagtttggctttaacattcgtgagggtgcaaatcagaagacggtcgtgggttataaggcaagcgatagtcgtatgtttgtggaccgaactgcatccggtgaaacggatttttctaaccttttcagtaagaaacatgaagcgcctacgcaaatggagaataatcggatcaagatgcgtattcttgtggatgagtcttctgttgaagcctttggtaacgacggcaaagtcgtcttttcagatgttatatttccggatcctgctagtagagcgatgagtttttacgtaaaaggcgggaatgtgaatgttgtttccttgaaagtgcatcaacttcaatctgtctggaatgcggacattccttcaaaagttcaaataaagatggatacgagcgttcgggaactgggggtaggcgagtcggatacactgcaggcgatggtcgagtatggcccgggtctaggcgttcaaccgcttaagtggaatccagtaataacgacg

SEQ ID NO: 2 - protein, 923 aa, Paenibacillus amylolyticus
MKDKSKFAICLLMATGLTLTSNGIFTSKSIAASAVLADQETNKQAKEELDIFESASKSNTNLIGWQVKGKGGLEDTSEGILLTSQPKENVMAISETVSDDFIYEADVMIRDTKADATLLFRSNEDGFSSYMLQIVPDAGLIRLRDARTGDGKLKEERKVSVEMGQIYHLKVKAVGSSLKVYWGNQYKPLIDVQDISYQSGKLGLHVWDGSALFSNIVVSDLKGNLGTVLSSKGKWQPDINGKRGTVEQGSIAQQIYNKEATDMVYEGNITLRPDSIAALAFRSSTDGAEGYEATLTKEGDQVRVSLTNTKGTVIASSQRTYPSQMGAKHHVEIKAKGDRIQVFLDGYTTAAIDVKDSTYKSGSTGIVVKKGTAYFQDTYVTELSQYYNEIYRPQYHYTPIRGSASDPNGLVYFEGEYHLFHQDGGTWAHAVSKDMLNWKRLPIALPWNDHGHVWSGSAVADMTNASGLFGDSGGKGLIAYYTSFNPDSPNGNQRIGLAYSKDQGRTWEYSKERPIVIENPGKSGNEAGNWDFRDPKVIRDDENNRWVMVVSGGDHIRFYTSTNLLDWTLTDNWGYGDYVRGGVWECPDLFQLPVDGTSQKKWVMMISTGANPKTGGSDAEYFIGHLTADGKFVNDNPAGKVLRTDFGKEFYASMSFANMPDHRTVMMAWMTNWDYPFAFPTSNWKGELTIPREVSLVTTEDGIRMVQSPIKELESLRKPLYSASNKSVSPSSGNLLKGIISGAYEIEAEIEIPETSTVTEFGFNIREGANQKTVVGYKASDSRMFVDRTASGETDFSNLFSKKHEAPTQMENNRIKMRILVDESSVEAFGNDGKVVFSDVIFPDPASRAMSFYVKGGNVNVVSLKVHQLQSVWNADIPSKVQIKMDTSVRELGVGESDTLQAMVEYGPGLGVQPLKWNPVITT

SEQ ID NO: 3 - protein, 892 aa, Paenibacillus amylolyticus
ASAVLADQETNKQAKEELDIFESASKSNTNLIGWQVKGKGGLEDTSEGILLTSQPKENVMAISETVSDDFIYEADVMIRDTKADATLLFRSNEDGFSSYMLQIVPDAGLIRLRDARTGDGKLKEERKVSVEMGQIYHLKVKAVGSSLKVYWGNQYKPLIDVQDISYQSGKLGLHVWDGSALFSNIVVSDLKGNLGTVLSSKGKWQPDINGKRGTVEQGSIAQQIYNKEATDMVYEGNITLRPDSIAALAFRSSTDGAEGYEATLTKEGDQVRVSLTNTKGTVIASSQRTYPSQMGAKHHVEIKAKGDRIQVFLDGYTTAAIDVKDSTYKSGSTGIVVKKGTAYFQDTYVTELSQYYNEIYRPQYHYTPIRGSASDPNGLVYFEGEYHLFHQDGGTWAHAVSKDMLNWKRLPIALPWNDHGHVWSGSAVADMTNASGLFGDSGGKGLIAYYTSFNPDSPNGNQRIGLAYSKDQGRTWEYSKERPIVIENPGKSGNEAGNWDFRDPKVIRDDENNRWVMVVSGGDHIRFYTSTNLLDWTLTDNWGYGDYVRGGVWECPDLFQLPVDGTSQKKWVMMISTGANPKTGGSDAEYFIGHLTADGKFVNDNPAGKVLRTDFGKEFYASMSFANMPDHRTVMMAWMTNWDYPFAFPTSNWKGELTIPREVSLVTTEDGIRMVQSPIKELESLRKPLYSASNKSVSPSSGNLLKGIISGAYEIEAEIEIPETSTVTEFGFNIREGANQKTVVGYKASDSRMFVDRTASGETDFSNLFSKKHEAPTQMENNRIKMRILVDESSVEAFGNDGKVVFSDVIFPDPASRAMSFYVKGGNVNVVSLKVHQLQSVWNADIPSKVQIKMDTSVRELGVGESDTLQAMVEYGPGLGVQPLKWNPVITT

SEQ ID NO: 4 - DNA, 1539 bp, Aspergillus deflectus
  CDS (1)..(1536)
  sig_peptide (1)..(48)
  mat_peptide (49)..(1536)
atgctcctgtctctctcattgattgcacttgccacgcaagctgtggccgacgacttccgccccgtcttccacttcgtccccgagaagaactggatgaacgagcccaacggcttgatccagatcgactccgtctggcacctgttctaccagcacaacccaaccgcgaacgtctggggcaatctgaactggggccacgcaaccagcagtgacctagtccactggactcacgagcccctcgcaatcaccagcgagaacggcgtcgaagccttcaccggcaccgccttttacgacgccgagaatacctctggtctcggatctgccgccagtccgccgtacctggcgtggtacactgggtatctcccgtcgaatgggacccaggaccagcgtcttgcctttagtatcgatgacggtgtaacctggaccaagttccagggcaatcccatcatctcagccgcgcaggagacaccacatgacgagacggccgggctggaaagccgcgatcccaaggtcttcttccatgaaccgtccgggaaatgggtcatggtgctcgcgcatggaggacaggacaagttgaccttctggacgtcaacggacgccatatcctggacgtggaagagcgatctttcggcggaccaggtcgatggtctgccttctgacatcaccgggtgggaggtgccggatatgttcgagctccccatcgagggcaccgacgagaccacctgggtgctgattatgactcccgcagaggggtccccagccggtgggaatggcgtcctcgccttgacgggctccttcgatgggtctgtcttcacgcctgaccctgtcgaccctgcgtcttctggcctttggctcgactacggtcgtgacttcgacggcgccatgagctgggagaatgtgcctgccgcagatggacggcggatcctggcctccgtcatgaatagctacggggcaagcccgccgaccaacacctggaaagggatgctgtcgttcccgcgcacgatcgcattgagggaaacagacaccaagaagagatacttccttcaacagcctgtcaccgaacttagctccgtcagctcctctcctctcgcgacaatcaagaatcagaccgtcacgcccggccaaaccttgctgtcgtcggttcacggcaccgcgctggatatcaaactggccttctccatgaacgaaggcgccaccctcgccctggcagtgcgcaaagcagcctcggagcagacggtcatctcgtacagccagtcgaacgggaccttgtccgtggaccgcacggcgagtgggaatacctcgtacgaccctgcggccggcggagtccacagcacgtcgctgcagcccgacgactctggagtggttcacatccgggttctggttgacacttgctcggtggaggtctttggaggggacggcgaggtggtgatttcagacctggtcttccccagcgagacctctgatgggctgtccctgcaggtgtctgggggggtggccaatctacacttggtcgaggtgcgcggaatttcgctccaggagtaa

SEQ ID NO: 5 - protein, 512 aa, Aspergillus deflectus
MLLSLSLIALATQAVADDFRPVFHFVPEKNWMNEPNGLIQIDSVWHLFYQHNPTANVWGNLNWGHATSSDLVHWTHEPLAITSENGVEAFTGTAFYDAENTSGLGSAASPPYLAWYTGYLPSNGTQDQRLAFSIDDGVTWTKFQGNPIISAAQETPHDETAGLESRDPKVFFHEPSGKWVMVLAHGGQDKLTFWTSTDAISWTWKSDLSADQVDGLPSDITGWEVPDMFELPIEGTDETTWVLIMTPAEGSPAGGNGVLALTGSFDGSVFTPDPVDPASSGLWLDYGRDFDGAMSWENVPAADGRRILASVMNSYGASPPTNTWKGMLSFPRTIALRETDTKKRYFLQQPVTELSSVSSSPLATIKNQTVTPGQTLLSSVHGTALDIKLAFSMNEGATLALAVRKAASEQTVISYSQSNGTLSVDRTASGNTSYDPAAGGVHSTSLQPDDSGVVHIRVLVDTCSVEVFGGDGEVVISDLVFPSETSDGLSLQVSGGVANLHLVEVRGISLQE

SEQ ID NO: 6 - protein, 496 aa, Aspergillus deflectus
DDFRPVFHFVPEKNWMNEPNGLIQIDSVWHLFYQHNPTANVWGNLNWGHATSSDLVHWTHEPLAITSENGVEAFTGTAFYDAENTSGLGSAASPPYLAWYTGYLPSNGTQDQRLAFSIDDGVTWTKFQGNPIISAAQETPHDETAGLESRDPKVFFHEPSGKWVMVLAHGGQDKLTFWTSTDAISWTWKSDLSADQVDGLPSDITGWEVPDMFELPIEGTDETTWVLIMTPAEGSPAGGNGVLALTGSFDGSVFTPDPVDPASSGLWLDYGRDFDGAMSWENVPAADGRRILASVMNSYGASPPTNTWKGMLSFPRTIALRETDTKKRYFLQQPVTELSSVSSSPLATIKNQTVTPGQTLLSSVHGTALDIKLAFSMNEGATLALAVRKAASEQTVISYSQSNGTLSVDRTASGNTSYDPAAGGVHSTSLQPDDSGVVHIRVLVDTCSVEVFGGDGEVVISDLVFPSETSDGLSLQVSGGVANLHLVEVRGISLQE

SEQ ID NO: 7 - DNA, 1551 bp, Aspergillus niger
  CDS (1)..(1548)
  sig_peptide (1)..(75)
  mat_peptide (76)..(1548)
atgttgaatccgaaggttgcctacatggtctggatgacgtgcctgggtttaacgttgcccagccaggcacagtctaatgattaccgtccttcataccacttcacaccggaccagtactggatgaacgagccaaacggcctgattaaaatcggatccacctggcacctgttctttcaacacaatccgacggccaatgtatggggcaacatatgctgggggcacgctacgagcaccgatctgatgcactgggcacacaaacccactgccattgcggatgagaacggagtcgaagcgtttaccggtacagcctattatgatccaaacaatacctctggccttggggattcggcaaacccaccctatctggcctggttcacaggttataccacttcaagccaaacacaggaccagcgcctggctttcagtgtggataacggggcgacgtggaccaaatttcaaggcaatcccatcatatcaactagccaggaagcaccacatgatataacgggcggcctcgagagtagggatccaaaggtattcttccatcgccaatcggggaactggatcatggttctcgcccatggcgggcaggacaagctgtctttctggacgtctgcagacaccataaactggacatggcagagtgacctgaagtccacctcgatcaatggcctatcgtccgatattacagggtgggaagtccccgacatgtttgaactcccggttgaaggcactgaggagaccacgtgggtggtgatgatgacgccggctgaaggatcccctgccggtggtaacggggtcttagctatcaccggttcttttgacgggaaaagttttacggcagatcccgtcgatgcttcgaccatgtggctggacaatgggcgtgatttcgatggcgctctgagctgggtgaacgtgcctgcgtccgatggacggcggattatcgccgccgtcatgaatagctacggttccaacccgcctacaaccacctggaaagggatgctctcctttccccggacgctgtcgctcaagaaagttggcacgcagcagcactttgttcaacagccgatcacagagttggatacaatcagtaccagtctgcaaatactagcaaaccagaccattacccctggccaaacattgctgtcatcgattcggggaactgctctcgatgttcgagttgctttttaccctgatgctggctcggttctgtccctcgccgtccgaaagggtgcttcggagcaaacagtcattaagtacacccagtcagatgccacattgtcggttgatcgaacagagagtggagatatctcgtatgacccggccgcaggtggcgtccataccgccaagttggaagaggacggcaccggactggtttccatccgggtgttggtggatacgtgttctgtagaggtttttggcggacaaggagaagccgtcatttccgacctcatcttcccgagtgacagttctgacggcctggccttggaggtaactggcggaaatgcagtgctgcagtcggtggacgtgcggagtgtttcacttgaatga

SEQ ID NO: 8 - protein, 516 aa, Aspergillus niger
MLNPKVAYMVWMTCLGLTLPSQAQSNDYRPSYHFTPDQYWMNEPNGLIKIGSTWHLFFQHNPTANVWGNICWGHATSTDLMHWAHKPTAIADENGVEAFTGTAYYDPNNTSGLGDSANPPYLAWFTGYTTSSQTQDQRLAFSVDNGATWTKFQGNPIISTSQEAPHDITGGLESRDPKVFFHRQSGNWIMVLAHGGQDKLSFWTSADTINWTWQSDLKSTSINGLSSDITGWEVPDMFELPVEGTEETTWVVMMTPAEGSPAGGNGVLAITGSFDGKSFTADPVDASTMWLDNGRDFDGALSWVNVPASDGRRIIAAVMNSYGSNPPTTTWKGMLSFPRTLSLKKVGTQQHFVQQPITELDTISTSLQILANQTITPGQTLLSSIRGTALDVRVAFYPDAGSVLSLAVRKGASEQTVIKYTQSDATLSVDRTESGDISYDPAAGGVHTAKLEEDGTGLVSIRVLVDTCSVEVFGGQGEAVISDLIFPSDSSDGLALEVTGGNAVLQSVDVRSVSLE

SEQ ID NO: 9 - protein, 491 aa, Aspergillus niger
NDYRPSYHFTPDQYWMNEPNGLIKIGSTWHLFFQHNPTANVWGNICWGHATSTDLMHWAHKPTAIADENGVEAFTGTAYYDPNNTSGLGDSANPPYLAWFTGYTTSSQTQDQRLAFSVDNGATWTKFQGNPIISTSQEAPHDITGGLESRDPKVFFHRQSGNWIMVLAHGGQDKLSFWTSADTINWTWQSDLKSTSINGLSSDITGWEVPDMFELPVEGTEETTWVVMMTPAEGSPAGGNGVLAITGSFDGKSFTADPVDASTMWLDNGRDFDGALSWVNVPASDGRRIIAAVMNSYGSNPPTTTWKGMLSFPRTLSLKKVGTQQHFVQQPITELDTISTSLQILANQTITPGQTLLSSIRGTALDVRVAFYPDAGSVLSLAVRKGASEQTVIKYTQSDATLSVDRTESGDISYDPAAGGVHTAKLEEDGTGLVSIRVLVDTCSVEVFGGQGEAVISDLIFPSDSSDGLALEVTGGNAVLQSVDVRSVSLE

SEQ ID NO: 10 - DNA, 4352 bp, Penicillium ochrochloron
  CDS (1)..(399)
  sig_peptide (1)..(63)
  mat_peptide (64)..(399)
  CDS (453)..(4349)
atggcttctcggcttgtgacagccttgactctgggtcttcatttattccaagcagcatttgctcaaacctacgatgagctctaccgccctcagtatcactttaccccccctgtgaactggatgaatgatcccaacggacttttatatcacaatggcctctatcatctgtactatcaatacaacccaggagggaacacctggggagctatgtcttggggccatgccactagcactgatctgactcactggaatcaccagcctattgccctcctcgctcgtggctacccaggtgatatcactgagatgttcttctctggctctgctgtcgccgatactcagaacacgagtggatttggtactagtggaaaggtgccatttgttgcgatgtatacttcttatgtgagtatttgaaacctttaaagtcggctactctaaccctttttcggccttagtaccctgcatcccagaacctccccagtgggaagttagtcaacgggggccagcaagcgcagtcaattgcctacagtttggacgaaggcatgacatggacaacctacgacgccgccaatcctgtcattctcaatccccctacgccatatacagaccaatggcgggacttccgagatccattcctgttctggcacgaagccagccagaagtggatctctgtggtttcactggcccaactccacaaattacttatttacacctccccgaatcttaaggattggacttattcaagcgaatttggaccttggaatgcagtcgggggtgtttgggagtgtcctagcctctttccacttgctgttgacggagatgacgccaatacaaaatggattatgcaaatcgggctgaaccctggcgggccccctggagtgactggctcaggaacgcagtatattgtgggaacctttgatggaacaaaatttgttgcggatcccaactctccactttcggcacatacatccaccagtaccactacctcagcatccgtcactacctcagcatccatcactacctcagcatccatcactcccacaaccacaaaagcaactgcgactgctactgcaactggagacatcatctttcaggattttgaaggcgccggtgacttcgcctctcgtggctgggttgccaccggtggtttgcttggggctgctccagctaaagggactcttgcgggacagcaaaccgtcactggatatgctggaagccagctggtcaataccttcctaagcggagactctacaactggcacgctcacatccccggctttcactatatcacttccatatgtcaatttcctaattggcggcgggaatgctccagggacggaatgcatcaacctgaaggtgcaaggccaggctgtccggacggcgacaggcgcgaacgtggaacaactcgcaccaacaacctggaatgtcacggacctgatgggtcaaaccgctgtgcttgagatcgtggatcagcaaacaggtggctggggacacatattgattgatcagattaccttcaccagcagtaccagcaccaacaacctccacaaacgcagcgtttctagcgttacttgggatttcaatggtactagcacttttgcagactatggctggactccgtctggagattttgttggaaaagggccagcccagggcacacttgcgggacaacaggttgtgactgggaatatgggaaacttcgtcaacacatttttgagcggagatggtaccactggaacactcacgtcccccgccttcaccataactcagaagaagattaactttctcatcggtggaggcaattctccgggcatggaatgcatcaatctcaagatccaaggccaggttgtacgaacggctacaggcgctaatgcagaacaactcgtaccaaccacctgggatgtcaccgacctgataggccagtctgcggtcatcgagattgttgatctcagtacgaccggctgggggcacattctgatcgatgagatttccttctcaaacatatcaattgtgccatatgggcccaactggttggactatgggccggacttctacgctgcaacaacattcaatggattatcatccacaaatcgaatcgatattgcgtggatgaacaattggcaatacgctagcgcaatccctacttctccatggcgcagcatgctttcagttgcccggaagctttcactcaagacgatcgacgacaggccaagactgatccagcaacctgaggcaaactggacgagtctacagacaacaacgtattctaccagctttgatacagttgctgaagggaatcagctcgtacaactctccggaaaattgcttgacatcacagtggccttttcagacagagttgcagcgtcgtcctcatcgcaattcggcataattctcagagcaacatctgacctagcacagcaaacacggattggctatgattttagcacaaagaggctttttgttgatcgaacgaaatctggaaatgttggcttcgacgggacttttcccagcacctattttgctcctttggcaccctccggtgatggccgagtcacaatgcgcattcttcttgacaggtcctccgttgaagtctttggaggccagggtgaggtcacaatatccgctcaaatctttccgcaggacaatggtgttgatgtccggcttttctccagaggcgggagtacaaatgctatcacgattgatgcaattgtgcttgattcagtttatgattcatccaccccaagtgcttctctcagtacgagctcaacctccatcggtactgtcaccaccaccttcagtaacaccgtgactacctctgtcggtaacaccctgaccaccagtacaagaaccgtcactagcgctagccctacgtctacagcgccctacgattttcgtcctgtttttcattttgtgccagaagagaactggatgaatgagcctaacggactgatcaaaattgactctacctggcaccttttctttcaacacaacccaactggaaacttttggggcaacttaagttggggtcatgctactagcattgaccttgtgtcctgggattataaaccggttgcgatttcaagcgcagatgggattcaggctttcacaggaacctcatactttgatgcgatgaatctctcggggcttgggacatcatcaaaccagccgtaccttgccttttatactggctacgcaccttcaagtggcatacaggatcaaaggcttgcgtatagcttggaccagggagcgacatggatgaaataccagggtaatccaattatatcccaaagccaagaagcgccacacgatataacccagggtttggagatacgtgatccaaaggtgttctaccacagtccgacgagcagatgggttatgatcctggcgcacggaggacaaaacaaactaacattctggacgtctaccgatgcaaagaactggacctggagaagtaatttcgctgcaagcaatattcttggattccctagtggggtcaacggttgggaagtgccagactttttcgaacttcggattcaaggtactacgcaatataaatgggtgttgattgtgacccccgccgctggatcgcctgctggtggtaatggggtctttgcactcaccggatctttcgacggcgaggtatttacagcagacgcggttgaccctaccactctatggctcgattatggtcgcgattgggatggggtaatgagttgggaaaacgtgcctgcttcggacgggcgcagggttcttgctgcagtaatgaacagttatggtggcaaccccccaactaatacgtggaagggaatgctttcattccctcgtactctggaactcaagcaacttaatggcaaactccgattcctccaattgcccgtgagcgaactagacgcggtcggtgcttcagttgcgactatcacgaatcagactcttgcacctggacaaacattgctctctaacatccattcccggtcattggacatccttattacgtttgttccagcccagggctctacactgtctctctccgttcggaagggagggtctcaacaaaccgtgattgaatatgtccagtccaacaaccaactttcggtcgatcgcaatgaaagtggaaacacctcatacgatcctgcagctggcggtgtccacagcgccgctctccacgctgatgagaatgggaaggtgcaattgcgagtattggttgatgaatgctccattgaggtctatggcgggcaaggggaggcggtgatctctgatttgatattccccgatttttcctcggatggcctggctttgtccactagtcaagggaacgtggtattggaatcagtcgacgtgcggtcgattttgctctga

SEQ ID NO: 11 - protein, 1432 aa, Penicillium ochrochloron
MASRLVTALTLGLHLFQAAFAQTYDELYRPQYHFTPPVNWMNDPNGLLYHNGLYHLYYQYNPGGNTWGAMSWGHATSTDLTHWNHQPIALLARGYPGDITEMFFSGSAVADTQNTSGFGTSGKVPFVAMYTSYYPASQNLPSGKLVNGGQQAQSIAYSLDEGMTWTTYDAANPVILNPPTPYTDQWRDFRDPFLFWHEASQKWISVVSLAQLHKLLIYTSPNLKDWTYSSEFGPWNAVGGVWECPSLFPLAVDGDDANTKWIMQIGLNPGGPPGVTGSGTQYIVGTFDGTKFVADPNSPLSAHTSTSTTTSASVTTSASITTSASITPTTTKATATATATGDIIFQDFEGAGDFASRGWVATGGLLGAAPAKGTLAGQQTVTGYAGSQLVNTFLSGDSTTGTLTSPAFTISLPYVNFLIGGGNAPGTECINLKVQGQAVRTATGANVEQLAPTTWNVTDLMGQTAVLEIVDQQTGGWGHILIDQITFTSSTSTNNLHKRSVSSVTWDFNGTSTFADYGWTPSGDFVGKGPAQGTLAGQQVVTGNMGNFVNTFLSGDGTTGTLTSPAFTITQKKINFLIGGGNSPGMECINLKIQGQVVRTATGANAEQLVPTTWDVTDLIGQSAVIEIVDLSTTGWGHILIDEISFSNISIVPYGPNWLDYGPDFYAATTFNGLSSTNRIDIAWMNNWQYASAIPTSPWRSMLSVARKLSLKTIDDRPRLIQQPEANWTSLQTTTYSTSFDTVAEGNQLVQLSGKLLDITVAFSDRVAASSSSQFGIILRATSDLAQQTRIGYDFSTKRLFVDRTKSGNVGFDGTFPSTYFAPLAPSGDGRVTMRILLDRSSVEVFGGQGEVTISAQIFPQDNGVDVRLFSRGGSTNAITIDAIVLDSVYDSSTPSASLSTSSTSIGTVTTTFSNTVTTSVGNTLTTSTRTVTSASPTSTAPYDFRPVFHFVPEENWMNEPNGLIKIDSTWHLFFQHNPTGNFWGNLSWGHATSIDLVSWDYKPVAISSADGIQAFTGTSYFDAMNLSGLGTSSNQPYLAFYTGYAPSSGIQDQRLAYSLDQGATWMKYQGNPIISQSQEAPHDITQGLEIRDPKVFYHSPTSRWVMILAHGGQNKLTFWTSTDAKNWTWRSNFAASNILGFPSGVNGWEVPDFFELRIQGTTQYKWVLIVTPAAGSPAGGNGVFALTGSFDGEVFTADAVDPTTLWLDYGRDWDGVMSWENVPASDGRRVLAAVMNSYGGNPPTNTWKGMLSFPRTLELKQLNGKLRFLQLPVSELDAVGASVATITNQTLAPGQTLLSNIHSRSLDILITFVPAQGSTLSLSVRKGGSQQTVIEYVQSNNQLSVDRNESGNTSYDPAAGGVHSAALHADENGKVQLRVLVDECSIEVYGGQGEAVISDLIFPDFSSDGLALSTSQGNVVLESVDVRSILL

SEQ ID NO: 12 - protein, 1411 aa, Penicillium ochrochloron
QTYDELYRPQYHFTPPVNWMNDPNGLLYHNGLYHLYYQYNPGGNTWGAMSWGHATSTDLTHWNHQPIALLARGYPGDITEMFFSGSAVADTQNTSGFGTSGKVPFVAMYTSYYPASQNLPSGKLVNGGQQAQSIAYSLDEGMTWTTYDAANPVILNPPTPYTDQWRDFRDPFLFWHEASQKWISVVSLAQLHKLLIYTSPNLKDWTYSSEFGPWNAVGGVWECPSLFPLAVDGDDANTKWIMQIGLNPGGPPGVTGSGTQYIVGTFDGTKFVADPNSPLSAHTSTSTTTSASVTTSASITTSASITPTTTKATATATATGDIIFQDFEGAGDFASRGWVATGGLLGAAPAKGTLAGQQTVTGYAGSQLVNTFLSGDSTTGTLTSPAFTISLPYVNFLIGGGNAPGTECINLKVQGQAVRTATGANVEQLAPTTWNVTDLMGQTAVLEIVDQQTGGWGHILIDQITFTSSTSTNNLHKRSVSSVTWDFNGTSTFADYGWTPSGDFVGKGPAQGTLAGQQVVTGNMGNFVNTFLSGDGTTGTLTSPAFTITQKKINFLIGGGNSPGMECINLKIQGQVVRTATGANAEQLVPTTWDVTDLIGQSAVIEIVDLSTTGWGHILIDEISFSNISIVPYGPNWLDYGPDFYAATTFNGLSSTNRIDIAWMNNWQYASAIPTSPWRSMLSVARKLSLKTIDDRPRLIQQPEANWTSLQTTTYSTSFDTVAEGNQLVQLSGKLLDITVAFSDRVAASSSSQFGIILRATSDLAQQTRIGYDFSTKRLFVDRTKSGNVGFDGTFPSTYFAPLAPSGDGRVTMRILLDRSSVEVFGGQGEVTISAQIFPQDNGVDVRLFSRGGSTNAITIDAIVLDSVYDSSTPSASLSTSSTSIGTVTTTFSNTVTTSVGNTLTTSTRTVTSASPTSTAPYDFRPVFHFVPEENWMNEPNGLIKIDSTWHLFFQHNPTGNFWGNLSWGHATSIDLVSWDYKPVAISSADGIQAFTGTSYFDAMNLSGLGTSSNQPYLAFYTGYAPSSGIQDQRLAYSLDQGATWMKYQGNPIISQSQEAPHDITQGLEIRDPKVFYHSPTSRWVMILAHGGQNKLTFWTSTDAKNWTWRSNFAASNILGFPSGVNGWEVPDFFELRIQGTTQYKWVLIVTPAAGSPAGGNGVFALTGSFDGEVFTADAVDPTTLWLDYGRDWDGVMSWENVPASDGRRVLAAVMNSYGGNPPTNTWKGMLSFPRTLELKQLNGKLRFLQLPVSELDAVGASVATITNQTLAPGQTLLSNIHSRSLDILITFVPAQGSTLSLSVRKGGSQQTVIEYVQSNNQLSVDRNESGNTSYDPAAGGVHSAALHADENGKVQLRVLVDECSIEVYGGQGEAVISDLIFPDFSSDGLALSTSQGNVVLESVDVRSILL

SEQ ID NO: 13 - DNA, 1524 bp, Penicillium murcianum
  CDS (1)..(1521)
  sig_peptide (1)..(45)
  mat_peptide (46)..(1521)
atgttcctcgccttagcattcgcgctcgccattcgggtaatggcggatgacttccgcccagtctaccattttgtccctaaggagaactggatgaacgaacccaatgggttgattaagatcaaatcaacctggcacctgttctatcaacacaacccaacggcaaatgtctggggcaatctgaactggggccacgcaaccagtagtgacctggtccactggactcacgagccccttgccataataagcgagaacggtgtcgaagcgttcaccggcactagctattatgatgccgacaacacctccgctctaggatcttctaccagcccaccctatctggcctactacactgggtattttccttctaacgggacccaggaccagcgtcttgcctttagtgtcgacgatggcgtgacctggacgaagtaccagggcaatcctattatctcagccgctcaggagtcgccgcatgataagacggcggggctggagacccgcgaccctaaggtcttttttcaccaggcatctgggaaatgggtcatggtccttgcccatggcggacaggacaagctaacgttttggacgtcgtcgaacgcaaagtcctggacgtggaagagcgacctctctgctagccagatcgagggtctaccctctggaattactgggtgggaagtgcctgatatgttcgaactccctgtccaggacactaatgcgaccacctgggtgttaattataacccctgcaaacggatctcccgctggtggtaatggtgttctagccctgacaggctcctttgatggatcaacattcaaacctgaccctgtcgacccttctgctctttggctcgactatggtcgcgatttcgacggtgctttgagttgggaaaatgtccctgctgcagatagtcggcggcggatccttgcctctgtcatgaacagctacggcgctagcccaccaaccaatacctggaaagggatgctgtctttccctcggaccattaccctaaaggaaacagcctctaagcgatacttcctccagcagcctgtcactgagctctccctcgttggttcctcgttagtgacaatccagaatcaaacaattaccaccggccaaacgttgctgtcatccattcacggaaccgcgttagatatcaaaattgcctttaccattaacgcaggtgccaaactttcgctggcagtgcgcaaggccgcgtcagagcagacaatcatccagtatgaccagtcaaattcgtcgatatctgttgatcgaacgacgagtggagacatctcctacgaccccgctgcaggcggagtccacagtgcttccctgaaaccagatggttctggggttattcatctccgcgtcctagtcgacacctgctcggtggaggtctttggcggggaaggagaagttgtgatttcagacctgatttttcccagcgagacctccgacgggctgtccttggaggtaactgggggatcggccaatttgcactctgtcgaggtgcgcagcatttcgctcgaataa

SEQ ID NO: 14 - protein, 507 aa, Penicillium murcianum
MFLALAFALAIRVMADDFRPVYHFVPKENWMNEPNGLIKIKSTWHLFYQHNPTANVWGNLNWGHATSSDLVHWTHEPLAIISENGVEAFTGTSYYDADNTSALGSSTSPPYLAYYTGYFPSNGTQDQRLAFSVDDGVTWTKYQGNPIISAAQESPHDKTAGLETRDPKVFFHQASGKWVMVLAHGGQDKLTFWTSSNAKSWTWKSDLSASQIEGLPSGITGWEVPDMFELPVQDTNATTWVLIITPANGSPAGGNGVLALTGSFDGSTFKPDPVDPSALWLDYGRDFDGALSWENVPAADSRRRILASVMNSYGASPPTNTWKGMLSFPRTITLKETASKRYFLQQPVTELSLVGSSLVTIQNQTITTGQTLLSSIHGTALDIKIAFTINAGAKLSLAVRKAASEQTIIQYDQSNSSISVDRTTSGDISYDPAAGGVHSASLKPDGSGVIHLRVLVDTCSVEVFGGEGEVVISDLIFPSETSDGLSLEVTGGSANLHSVEVRSISLE

SEQ ID NO: 15 - protein, 492 aa, Penicillium murcianum
DDFRPVYHFVPKENWMNEPNGLIKIKSTWHLFYQHNPTANVWGNLNWGHATSSDLVHWTHEPLAIISENGVEAFTGTSYYDADNTSALGSSTSPPYLAYYTGYFPSNGTQDQRLAFSVDDGVTWTKYQGNPIISAAQESPHDKTAGLETRDPKVFFHQASGKWVMVLAHGGQDKLTFWTSSNAKSWTWKSDLSASQIEGLPSGITGWEVPDMFELPVQDTNATTWVLIITPANGSPAGGNGVLALTGSFDGSTFKPDPVDPSALWLDYGRDFDGALSWENVPAADSRRRILASVMNSYGASPPTNTWKGMLSFPRTITLKETASKRYFLQQPVTELSLVGSSLVTIQNQTITTGQTLLSSIHGTALDIKIAFTINAGAKLSLAVRKAASEQTIIQYDQSNSSISVDRTTSGDISYDPAAGGVHSASLKPDGSGVIHLRVLVDTCSVEVFGGEGEVVISDLIFPSETSDGLSLEVTGGSANLHSVEVRSISLE

SEQ ID NO: 16 - DNA, 2034 bp, Bacillus licheniformis
  CDS (1)..(2031)
  sig_peptide (1)..(72)
  mat_peptide (73)..(2031)
atgaaaaagagaatgattcagatggggatcataggggccatgatgtttccggaagcgttttctgcagctgcggccgacccggactactataacgaggatcatcggcccaagtatcatttcactccggaagcaaactggatgaatgacccgaacgggatggtgtattacgcgggggaatatcatctgttttatcaatatcatccgtatggcctgcgctgggggccgatgcactgggggcacgcagtaagcaaagatttggtcaagtgggagcatttgccggtcgcgctgtatccggatgaaaaagggacgattttttcgggcagcgctgtcgtcgatcggcataatacaacagggtttcaaacagggacggagaagccgctcgttgccatttatacgcaggaccgggacggggaacaagtacaaagcatcgcctacagcaatgacaaaggcaggacgtggacgaagtactcaggcaaccctgtcattccaaatccgggaaaaagagatttccgagatccgaaagtcgtttggcacaaacagacgaacaagtgggtgatgttgcttgcaggtggcgaccgaattctaatctatacatcgcctgacctgaagcagtggacatatgcgagcgaatttggaaaaggagaaggcagccatggaggcgtgtgggaatgtccggatctgtttgagcttcctgtagagggcagaccgaatgaaacgaaatgggtgatgcaagtcagtgttggagacggcgctgtctcaggaggatcaggcatgcaatatttcgtcggaagctttgacggaacaacctttaaaaatgaaaacccgccgaatcgcgtattatggacagattacggcaaagacttttacgcagctgtcagctggtctgatatcccgcagtcagacggacgaaggctttggttagggtggatgagcaattggcaatacgcaaacgatgttcccacttccccgtggagaagcgcaatgtctatcccgagagaactaaaattgaaagcgttttccgaggggttcagaattgttcaggctccggtagcggagctgaagtcgatcaggggtgcatcgcaaaggtggaaagacaagatcatttcgcctcgaaacagaaattttttgaaagcgctgtcaggtgatgcgtacgaaatcaatgcggaatttcaagtaaataccggaactgcggccgaattcggctttaaagtccgaacaggggaaaaccaatatacgaaaatcggatacagcaaaaacagcgcttccctcttcgtcgaccgaagccaatccggaaacgtctcctttcatccgaactttaacaccggaaagcatgcggccccgctgcaaccggtcggtgggaaggtgaagatgcgcatttatgtcgaccgctcttcagtcgaagtattcggcaacgacggaaggcaggtgatcaccgacattattctgccggaccgatccagcaaaggggttgaagcgtacgcatcaaacgggtttgtcaagctgaattcgatgacggttcatcctctgaaaaaagtatggggaacatcgccctttcaatcgaatttgaccggctggacgaccgtaaacggcgtatgggccgacacgattgatggaaaacaaggacgctctgacggggattcgttcattttatcttcagcaaaaggaacagatttcacatatgaagcagatgtcacggtaaaggatggaaatggcagaggggcgggtgctctcgtgtttcgggccgacaaggatgttcaaaacggataccttgccaatgtggatgccaagcatgatgtggtgaagtttttcaaatttgaaaacggctcggcctctgttattgccgaacaccgcacaccgctagaagccggccggaaatatcacctgaaggcggtggcccgcggttccaacttttatatttacttggatgaccgattggtgataagcgcccgggattccacgtttaccgagggagtattcggcttgaatgcatgggatgcgaccgcagtcttccaacatgtttatgctgatcgataa

SEQ ID NO: 17 - protein, 677 aa, Bacillus licheniformis
MKKRMIQMGIIGAMMFPEAFSAAAADPDYYNEDHRPKYHFTPEANWMNDPNGMVYYAGEYHLFYQYHPYGLRWGPMHWGHAVSKDLVKWEHLPVALYPDEKGTIFSGSAVVDRHNTTGFQTGTEKPLVAIYTQDRDGEQVQSIAYSNDKGRTWTKYSGNPVIPNPGKRDFRDPKVVWHKQTNKWVMLLAGGDRILIYTSPDLKQWTYASEFGKGEGSHGGVWECPDLFELPVEGRPNETKWVMQVSVGDGAVSGGSGMQYFVGSFDGTTFKNENPPNRVLWTDYGKDFYAAVSWSDIPQSDGRRLWLGWMSNWQYANDVPTSPWRSAMSIPRELKLKAFSEGFRIVQAPVAELKSIRGASQRWKDKIISPRNRNFLKALSGDAYEINAEFQVNTGTAAEFGFKVRTGENQYTKIGYSKNSASLFVDRSQSGNVSFHPNFNTGKHAAPLQPVGGKVKMRIYVDRSSVEVFGNDGRQVITDIILPDRSSKGVEAYASNGFVKLNSMTVHPLKKVWGTSPFQSNLTGWTTVNGVWADTIDGKQGRSDGDSFILSSAKGTDFTYEADVTVKDGNGRGAGALVFRADKDVQNGYLANVDAKHDVVKFFKFENGSASVIAEHRTPLEAGRKYHLKAVARGSNFYIYLDDRLVISARDSTFTEGVFGLNAWDATAVFQHVYADR

SEQ ID NO: 18 - protein, 653 aa, Bacillus licheniformis
ADPDYYNEDHRPKYHFTPEANWMNDPNGMVYYAGEYHLFYQYHPYGLRWGPMHWGHAVSKDLVKWEHLPVALYPDEKGTIFSGSAVVDRHNTTGFQTGTEKPLVAIYTQDRDGEQVQSIAYSNDKGRTWTKYSGNPVIPNPGKRDFRDPKVVWHKQTNKWVMLLAGGDRILIYTSPDLKQWTYASEFGKGEGSHGGVWECPDLFELPVEGRPNETKWVMQVSVGDGAVSGGSGMQYFVGSFDGTTFKNENPPNRVLWTDYGKDFYAAVSWSDIPQSDGRRLWLGWMSNWQYANDVPTSPWRSAMSIPRELKLKAFSEGFRIVQAPVAELKSIRGASQRWKDKIISPRNRNFLKALSGDAYEINAEFQVNTGTAAEFGFKVRTGENQYTKIGYSKNSASLFVDRSQSGNVSFHPNFNTGKHAAPLQPVGGKVKMRIYVDRSSVEVFGNDGRQVITDIILPDRSSKGVEAYASNGFVKLNSMTVHPLKKVWGTSPFQSNLTGWTTVNGVWADTIDGKQGRSDGDSFILSSAKGTDFTYEADVTVKDGNGRGAGALVFRADKDVQNGYLANVDAKHDVVKFFKFENGSASVIAEHRTPLEAGRKYHLKAVARGSNFYIYLDDRLVISARDSTFTEGVFGLNAWDATAVFQHVYADR

SEQ ID NO: 19 - protein, 182 aa, Bacillus cibi [sequence omitted]

SEQ ID NO: 20 - protein, 109 aa, Bacillus licheniformis
ARYDDILYFPASRYPETGAHISDAIKAGHSDVCTIERSGADKRRQESLKGIPTKPGFDRDEWPMAMCEEGGKGASVRYVSSSDNRGAGSWVGNRLSGFADGTRILFIVQ

SEQ ID NO: 21 - protein, 110 aa, Bacillus subtilis
ASSYDKVLYFPLSRYPETGSHIRDAIAEGHPDICTIDRDGADKRREESLKGIPTKPGYDRDEWPMAVCEEGGAGADVRYVTPSDNRGAGSWVGNQMSSYPDGTRVLFIVQ

SEQ ID NO: 22 - protein, 221 aa, Aspergillus oryzae
VPVNPEPDATSVENVALKTGSGDSQSDPIKADLEVKGQSALPFDVDCWAILCKGAPNVLQRVNEKTKNSNRDRSGANKGPFKDPQKWGIKALPPKNPSWSAQDFKSPEEYAFASSLQGGTNAILAPVNLASQNSQGGVLNGFYSANKVAQFDPSKPQQTKGTWFQITKFTGAAGPYCKALGSNDKSVCDKNKNIAGDWGFDPAKWAYQYDEKNNKFNYVGK

SEQ ID NO: 23 - protein, 188 aa, Trichoderma harzianum
APAPMPTPPGIPTESSARTQLAGLTVAVAGSGTGYSRDLFPTWDAISGNCNAREYVLKRDGEGVQVNNACESQSGTWISPYDNASFTNASSLDIDHMVPLKNAWISGASSWTTAQREALANDVSRPQLWAVSASANRSKGDRSPDQWKPPLTSFYCTYAKSWIDVKSFYKLTITSAEKTALSSMLDTC

SEQ ID NO: 24 - protein, 4 aa, Artificial Sequence
  Motif
  MISC_FEATURE (1)..(1): Xaa = Trp (W) or Pro (P) or Gly (G)
  MISC_FEATURE (2)..(2): Xaa = Gly (G) or Met (M) or Thr (T) or His (H)
  MISC_FEATURE (3)..(3): Xaa = Asn (N) or His (H)
  MISC_FEATURE (4)..(4): Xaa = Ala (A) or Glu (E) or Ile (I) or Leu (L) or Asp (D) or Asn (N) or Met (M) or Val (V)
XXXX

SEQ ID NO: 25 - protein, 6 aa, Artificial Sequence
  Motif
  MISC_FEATURE (2)..(2): Xaa = Tyr (Y) or Phe (F)
  MISC_FEATURE (4)..(4): Xaa = Leu (L) or Asn (N)
  MISC_FEATURE (6)..(6): Xaa = Gln (Q) or Lys (K)
AXSXDX

SEQ ID NO: 26 - protein, 6 aa, Artificial Sequence
  Motif
AYSNDK

SEQ ID NO: 27 - protein, 4 aa, Artificial Sequence
  Motif
  MISC_FEATURE (1)..(1): Xaa = His (H) or Ser (S)
XWGH

SEQ ID NO: 28 - protein, 8 aa, Artificial Sequence
  Motif
  MISC_FEATURE (1)..(1): Xaa = Gln (Q) or Leu (L) or Lys (K) or Glu (E) or Tyr (Y) or Met (M) or Arg (R) or Val (V) or Thr (T) or Ala (A) or Asn (N) or Ile (I)
  MISC_FEATURE (2)..(2): Xaa = Asn (N) or Ser (S) or Tyr (Y) or His (H) or Ala (A) or Thr (T) or Arg (R) or Asp (D)
  MISC_FEATURE (3)..(3): Xaa = Trp (W) or Phe (F)
  MISC_FEATURE (4)..(4): Xaa = Met (M) or Glu (E) or Val (V) or Lys (K) or Thr (T) or Leu (L)
  MISC_FEATURE (5)..(5): Xaa = Asn (N) or Gly (G) or Ala (A)
  MISC_FEATURE (6)..(6): Xaa = Glu (E) or Val (V) or Asp (D) or Leu (L) or Ile (I) or His (H)
  MISC_FEATURE (7)..(7): Xaa = Pro (P) or Ala (A) or Glu (E) or His (H) or Asp (D) or Ser (S)
  MISC_FEATURE (8)..(8): Xaa = Asn (N) or Ala (A) or Thr (T) or Ser (S) or Lys (K) or Gly (G) or Val (V) or Glu (E) or Arg (R) or Gln (Q) or Cys (C) or Asp (D)
XXXXXXXX

SEQ ID NO: 29 - protein, 8 aa, Artificial Sequence
  Motif
KNWMNEPN

SEQ ID NO: 30 - protein, 4 aa, Artificial Sequence
  Motif
  MISC_FEATURE (4)..(4): Xaa = Asp (D) or Glu (E)
WMNX

SEQ ID NO: 31 - protein, 7 aa, Artificial Sequence
  Motif
  MISC_FEATURE (4)..(4): Xaa = Asp (D) or Glu (E)
WMNXPNG

SEQ ID NO: 32 - protein, 4 aa, Artificial Sequence
  Motif
  MISC_FEATURE (2)..(2): Xaa = Gly (G) or Met (M) or Thr (T)
  MISC_FEATURE (4)..(4): Xaa = Asn (N) or Asp (D) or Glu (E) or Ile (I)
WXNX